(19) United States
(12) Patent Application Publication
(10) Pub. No.: US 2026/0176224 A1
(43) Pub. Date: Jun. 25, 2026

(54) Isomerization of trans-1,2-difluoroethylene (HFO-1132(E)) and/or cis-1,2-difluoroethylene (HFO-1132(Z))

(71) Applicant: Solstice Advanced Materials US, Inc., Morris Plains, NJ (US)

(72) Inventors: Yian Zhai, Lockport, NY (US); Dimitrios Papanastasiou, Buffalo, NY (US); Alexey Kruglov, Belmont, WA (US)

(21) Appl. No.: 19/412,833

(22) Filed: Dec. 8, 2025

Related U.S. Application Data
(60) Provisional application No. 63/736,426, filed on Dec. 19, 2024.

(51) Int. Cl. C07C 17/358

(52) U.S. Cl. CPC C07C 17/358

(57) ABSTRACT

A method for producing trans-1,2-difluoroethylene (HFO-1132(E)) by providing a reactant composition including cis-1,2-difluoroethylene (HFO-1132(Z)) and isomerizing the cis-1,2-difluoroethylene (HFO-1132(Z)) in a reactor at a temperature of from about 300° C. to about 600° C. in the presence of a catalyst to produce a product composition comprising HFO-1132(E).

### CROSS-REFERENCE TO RELATED APPLICATIONS

This application claims priority to U.S. Patent Application No. 63/736,426 entitled “Isomerization of trans-1,2-difluoroethylene (HFO-1132(E)) and/or cis-1,2-difluoroethylene (HFO-1132(Z))” filed on Dec. 19, 2024, the entire disclosure of which is incorporated by reference in its entirety.

### FIELD

The present disclosure is directed to methods for producing 1,2-difluoroethylene (HFO-1132), including trans-1,2-difluoroethylene (HFO-1132(E)) and/or cis-1,2-difluoroethylene (HFO-1132(Z)) and, in particular, to methods of conducting isomerization reactions between trans-1,2-difluoroethylene (HFO-1132(E)) and/or cis-1,2-difluoroethylene (HFO-1132(Z)).

### BACKGROUND

1,2-difluoroethylene (HFO-1132) has recently found increased utility for a variety of uses. HFO-1132 may exist as a mixture of two geometric isomers, the (E) or trans isomer and the (Z) or cis isomer, which may be used separately or together in various proportions. Potential end use applications of HFO-1132 include refrigerants, either used alone or in blends with other components, solvents for organic materials, and as a chemical intermediate in the synthesis of other halogenated hydrocarbon solvents.

Improved methods for the production of HFO-1132 and, in particular, HFO-1132(E), are desired.

### SUMMARY

The present disclosure provides methods of isomerizing cis-1,2-difluoroethylene (HFO-1132(Z)) and trans-1,2-difluoroethylene (HFO-1132(E)). The isomerization reactions may proceed through a catalyzed pathway or a non-catalyzed pathway. Advantageously, both the catalyzed and non-catalyzed pathways show high selectivity for the desired product (cis or trans) with minimal by-products.

In one form thereof, the present disclosure provides a method for producing trans-1,2-difluoroethylene (HFO-1132(E)), comprising: providing a reactant composition comprising cis-1,2-difluoroethylene (HFO-1132(Z)); and isomerizing the cis-1,2-difluoroethylene (HFO-1132(Z)) in a tubular reactor at a temperature of from about 300° C. to about 700° C. in the presence of a catalyst to produce a product composition comprising HFO-1132(E).

In another form thereof, the present disclosure provides a method for producing trans-1,2-difluoroethylene (HFO-1132(E)), comprising: providing a reactant composition comprising cis-1,2-difluoroethylene (HFO-1132(Z)); and isomerizing the cis-1,2-difluoroethylene (HFO-1132(Z)) in a semi-batch process within an autoclave reactor at a temperature of from about 300° C. to about 700° C. in the presence of a catalyst to produce a product composition comprising HFO-1132(E).

### BRIEF DESCRIPTION OF THE DRAWINGS

FIG. 1 is a schematic diagram of the apparatus used in Examples 1-4 for the isomerization reactions between HFO-1132(E) and/or HFO-1132(Z).

### DETAILED DESCRIPTION

### I. Definitions

As used herein, the singular forms “a”, “an” and “the” include plural unless the context clearly dictates otherwise. Moreover, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the disclosure be limited to the specific values recited when defining a range.

As used herein, the phrase “within any range encompassing any two of these values as endpoints” literally means that any range may be selected from any two of the values listed prior to such phrase regardless of whether the values are in the lower part of the listing or in the higher part of the listing. For example, a pair of values may be selected from two lower values, two higher values, or a lower value and a higher value. For example, a range of as low as 1, 2, or 3, or as high as 8, 9, or 10 followed by this phrase encompasses ranges including 1 to 10, or 2 to 8, or 3 to 9.

As used herein, the phrase “light impurities” refer to molecules that have lower boiling points than HFO-1132(E) or HFO-1132(Z).

As used herein, use of a “catalyst” in a “catalytic reaction” does not include irradiation, for example, ultraviolet radiation having a wavelength of from about 100 nm to about 400 nm, electromagnetic radiation having a wavelength of from about 380 nm to about 760 nm, infrared radiation having a wavelength of from about 780 nm to about 1 mm, and/or near-infrared radiation having a wavelength of from about 750 nm to about 2500 nm.

As used herein, the phrase “based on total amount of organic components of the composition” refers only to carbon-containing components and does not include or encompass non-carbon-containing components such as hydrogen (H₂) or hydrogen chloride (HCl).

As used herein, conversion of a reactant molecule (molecule X) during a reaction may be calculated using the following equation:

$$\% conversion of molecule X = ( 100 - molecule X wt . \% in the organic compo nen ts of a product mixture )$$

As used herein, selectivity to a molecule formed during a reaction (molecule X) may be calculated using the following equation:

$$\% selectivity to molecule X = wt . \% molecule X in the organic compo nen ts of a product mixture / ( 100 - wt . \% of reactant molecules in the organic compo nen ts of a product mixture ) \times 100.$$

As used herein, the phrase “once-through process” refers to a process during which a reactant composition is conveyed via a single pass through a reactor to form a product composition without further processing (e.g., going through a separation column or recycling).

As used herein, the phrase “once-through product” refers to a product composition collected and analyzed at the end of a once-through process.

As used herein, the phrase “integrated process” refers to a process during which a once-through product composition is then further processed in an integrated system, for example, going through a separation column for purification and/or recycling.

As used herein, the phrase “end product” refers to a product composition collected and analyzed at the end of an integrated process.

As used herein, calculation of the conversion and selectivity are based on the equivalency of a given molecule's gas chromatography (GC) percentage or wt. % in the mixture.

### II. Overview

The present disclosure provides a method for producing trans-HFO-1132(E) from cis-HFO-1132(Z) (“Process 1”), which includes providing a feed stream (e.g., reactant composition) containing a mixture of HFO-1132(E) and HFO-1132(Z), converting at least a portion of HFO-1132(Z) to HFO-1132(E), and separating and recovering HFO-1132(E) or a mixture of HFO-1132(E) and HFO-1132(Z). The reactor may be a tubular reactor including use of a catalyst, an autoclave reactor including use of a catalyst, or an autoclave reactor without use of a catalyst.

Schematic equation for Process 1 is represented below:

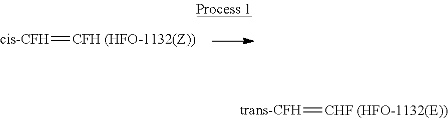

The present disclosure provides a method for producing cis-HFO-1132(Z) from trans-HFO-1132(E) (“Process 2”), which includes providing a feed stream containing a mixture of HFO-1132(E) and HFO-1132(Z), converting at least a portion of HFO-1132(E) to HFO-1132(Z), and separating and recovering HFO-1132(Z) or a mixture of HFO-1132(E) and HFO-1132(Z). The reactor may be a tubular reactor including use of a catalyst, an autoclave reactor including use of a catalyst, or an autoclave reactor without use of a catalyst.

Schematic equation for Process 2 is represented below:

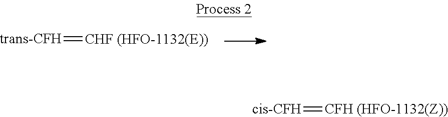

The feed stream of a mixture of HFO-1132(E) and HFO-1132(Z) may be obtained via various methods and starting materials.

For example, a mixture of HFO-1132(E) and HFO-1132(Z) may be obtained using CFCl₂—CF₂Cl (CFC-113) as a starting material via a two-step reaction process summarized below:

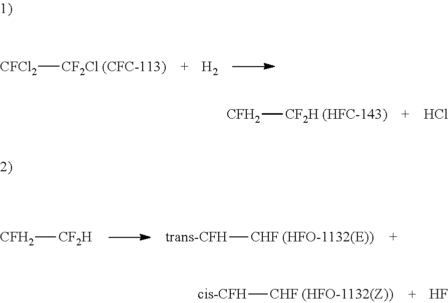

Alternatively, a mixture of HFO-1132(E) and HFO-1132(Z) may be obtained using chlorotrifluoroethylene (CTFE) or a mixture of CTFE and CF₂═CHF (HFO-1123) as starting materials via a two-step reaction process summarized below:

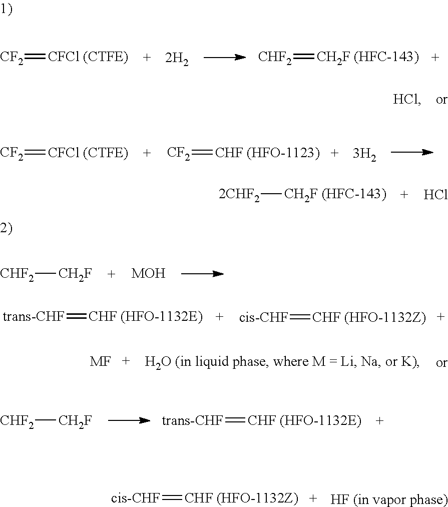

Alternatively, substantially pure HFO-1132(Z) may be used as the reactant composition for Process 1. Similarly, substantially pure HFO-1132(E) may be used as the reactant composition for Process 2.

Further details regarding each of the isomerization reactions of Process 1 and Process 2 are set forth below.

### III. Process 1

### General Process 1

As discussed above, a feed stream may include 1,2-difluoroethylene (HFO-1132) obtained via various methods or starting materials and including a mixture containing both the trans-HFO-1132(E) and cis-HFO-1132(Z) isomers, or substantially pure HFO-1132(Z).

In an integrated process, the cis-1,2-difluoroethylene HFO-1132(Z) isomer may be converted to the trans-1,2-difluoroethylene (HFO-1132(E)) isomer either by exposure to heat and/or a catalyst and further purified to form an end product comprising, consisting essentially of, or consisting of, the trans-1,2-difluoroethylene (HFO-1132(E)) isomer in high purity, such as at least about 95 wt. %, at least about 99.0 wt. %, at least about 99.9 wt. %, at least about 99.99 wt. % or greater, and for each of the foregoing, less than or equal to 100%, or within any range encompassed by two of the foregoing values as endpoints, based on total moles of the organic components of the end product composition.

The isomerization reaction may be conducted in a suitable reaction vessel or reactor. The reactor is preferably constructed from materials which are resistant to corrosion such as nickel and its alloys, including Hastelloy (for example, Hastelloy C276), Inconel (for example Inconel 600), and Monel. The reactor may also be a stainless steel reactor. The reactor may be made to include materials that may serve a similar function as a catalyst, but for a non-catalytic reaction.

The reactor may be first cleaned and flushed with an inert gas such as nitrogen. For catalytic processes, the reactor may then be packed with a catalyst such as those described below. The catalyst may be pretreated within the reactor such as by drying in the manner described further below, followed by metering the reactants into the reactor to initiate the reaction. The process flow may be in the down or up direction through a bed of the catalyst. Products may be flowed through one or more scrubbers to remove byproducts contained in the feed stream from the stream, such as hydrogen fluoride (HF) and/or hydrogen chloride (HCl), and the reaction products may be collected by capture in a cooled cylinder, for example.

### Process Flow (Process 1)

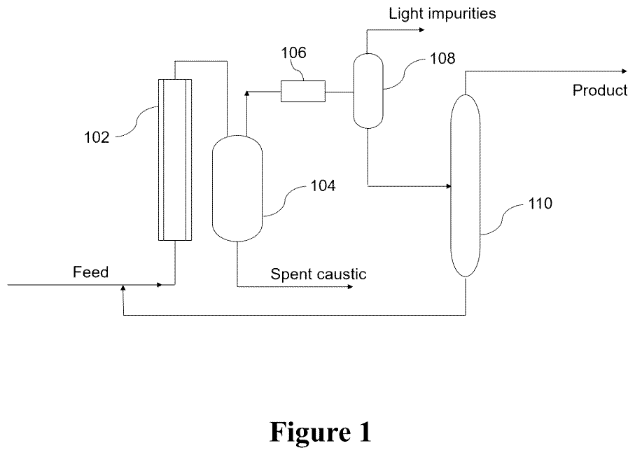

FIG. 1 includes a schematic process flow diagram illustrating suitable components for the reaction in Process 1 converting cis-1,2-difluoroethylene (HFO-1132(Z)) to trans-1,2-difluoroethylene (HFO-1132(E)). The process as shown in FIG. 1 may be used in isomerization reactions described below in Examples 1-4.

Referring to the process flow diagram shown in FIG. 1, a feed stream containing a mixture of HFO-1132(E) and HFO-1132(Z) or substantially pure HFO-1132(Z) (e.g., 99.9% 1132(Z)) is fed into a reactor 102 (e.g., a stainless steel reactor, or a Hastelloy reactor, for example, Hastelloy C276). The reactant stream may be vaporized by flowing through a series of heat exchangers (not shown) to a temperature above the boiling point of HFO-1132(Z) before being fed into the reactor 102. The reactor may be pre-heated to a desired reaction temperature by electricity or other heat sources, such as molten salt, etc.

The reactor 102 may be a tubular reactor. The flow rate may be from about 3 g/hr to about 40 g/hr, or from about 5 g/hr to about 35 g/hr, or from about 10 g/hr to 30 g/hr. The contact time may be from about 0.1 second to about 10 seconds, or from about 1 second to 8 seconds, or from about 1 second to about 6 seconds. The process through a tubular reactor may be a continuous flowing process with relatively short contact time. The effluent stream from the tubular reactor may then flow through a heat exchanger (not shown) and be further cooled down by a cooler (not shown) before entering the caustic scrubber 104 (e.g., a KOH scrubber) to remove impurities (e.g., HF). The overhead stream from the scrubber 104 may then go through a dryer 106 and collected in a liquid nitrogen trap (not shown) to obtain a product composition. The product composition may be expanded into a gas bag (e.g., a 1 L Tedlar gas bag), and analyzed by GC/GCMS.

Alternatively, the reactor 102 may be an autoclave reactor. The reactant (e.g., cis-1,2-difluoroethylene and/or trans-1,2-difluoroethylene) may be condensed into the autoclave reactor and sealed under vacuum. The contact time between the reactant composition and the reactor (and/or the catalyst in a catalytic process) may be from about 0.1 hour to about 1 hour, or from 0.3 hour to about 0.8 hour, or about 0.5 hour. The process through an autoclave reactor may be a batch like process (e.g., a semi-batch process) with longer holding time compared to a tubular reactor. The autoclave reactor may be heated to designated temperatures for the required duration. After a certain duration of contact time, the reactor may be cooled down to room temperature, and the content inside the autoclave reactor may be vacuum transferred into a cylinder (e.g., a 50 ml SS cylinder) to obtain a product composition. The product composition may optionally go through the scrubber 104 (e.g., a KOH scrubber) and/or the dryer 106 for the autoclave process before being transferred into the cylinder. The product composition may then be expanded into a gas bag (e.g., a 1 L Tedlar gas bag), and analyzed by GC/GCMS.

The reactor 102 (e.g., a tubular reactor or an autoclave reactor) can have a single wall and be heated with an external furnace or can be jacketed for heating with a circulating heat transfer medium, such as heating oil, thermal fluid, molten salt, etc. to heat at least a portion of the reactant composition during the reaction.

In an integrated process, where the product composition is not collected for analysis, the product composition may enter the light knockout kettle or knockout drum 108 to further remove impurities, for example light impurities (e.g., ethane, ethylene, 1132a, vinyl chloride, vinyl fluoride, etc.). The condition of the knockout drum 108 is set to keep 1132(Z) and 1132(E) in the liquid phase and keep the light impurities in the vapor phase. For example, the drum 108 may be set to a temperature of about 25° C. and a pressure of 240 psig. The liquid stream from the drum 108 flows through a cooler and pressure regulating devices (e.g., pump, valve) to the bubble point at P=65 psig before feeding into a separation column 110. The separation column 110 may have 30 stages with an overhead pressure of about 60 psig.

The bottom stream from the column 110 may optionally be recycled back to the front end of the reactor 102 and combined with the feed stream. The bottom stream from column 110 may include, for example, a mixture of 1132(Z) and 1132(E). The top stream from the column 110 contains an end product containing substantially pure 1132(E).

### Catalysts for Process 1 (Tubular Reactor)

The catalyst and process conditions play an important role in the isomerization reaction. Suitable catalysts for using with a tubular reactor may include stainless steel (e.g., 316 stainless steel Pro-Pak®), metal oxides (e.g., chromium oxide, aluminum oxide), or metal alloys such as nickel alloy (e.g., Nickel Alloy 200 Pro-Pak®), nickel-chromium alloy (e.g., Nichrome Pro-Pak®), or alloys including nickel, chromium, manganese, iron, or a combination thereof (e.g., metal alloy having 1.5% manganese, 75%-78% nickel, 20%-23% chromium, and a balance of iron). Fluorination treatment of the catalyst may be conducted using anhydrous HF under conditions effective to convert a portion of metal oxides into corresponding metal fluorides, such as via the procedure disclosed in U.S. Pat. No. 6,780,815 to Cerri et al., the disclosure of which is expressly incorporated by reference herein. Other suitable catalysts for the catalytic reaction include metal fluorides such as chromium fluoride, alumina fluoride, iron fluoride, magnesium fluoride, and various combinations of thereof.

The catalyst used in Process 1 with a tubular reactor may have a proper BET (Brunauer, Emmet, and Teller) surface area. The BET analysis is the standard method for determining surface areas from nitrogen adsorption isotherms. The BET surface areas of catalysts may be measured using TriStar II Micromeritics instrument. Catalyst samples are degassed before the analysis using FlowPrep 060 instrument.

The BET surface area of the catalyst may be as low as about 1 m²/g, about 5 m²/g, about 10 m²/g, about 40 m²/g, about 50 m²/g, about 60 m²/g, about 70 m²/g, about 80 m²/g, about 90 m²/g, about 100 m²/g, or as high as about 110 m²/g, about 120 m²/g, about 130 m²/g, about 140 m²/g, about 150 m²/g, about 175 m²/g, about 200 m²/g, about 225 m²/g, about 250 m²/g, about 300 m²/g, or within any range encompassed by any of the foregoing values as endpoints, for example, from about 1 m²/g to about 2000 m²/g, about 10 m²/g to about 1000 m²/g, about 100 m²/g to about 800 m²/g, preferably from about 100 m²/g to about 500 m²/g, or more preferably from about 200 m²/g to about 500 m²/g.

For metal oxides catalysts, the BET surface area may be preferably between about 100 m²/g and about 200 m²/g. For fluorinated metal oxides catalysts, the BET surface area may be preferably between about 20 m²/g and about 2000 m²/g. For fluorinated alumina, the BET surface area may be greater than about 100 m²/g, preferably greater than 200 m²/g, most preferably greater than 250 m²/g, and for each of the foregoing, less than about 2000 m²/g, or within any range encompassed by any of the foregoing values as endpoints.

### Catalyst Pretreatment for Process 1 (Tubular Reactor)

The catalyst may be pretreated by drying at elevated temperatures, as low as about 200° C., about 250° C., about 300° C., about 350° C., about 360° C., about 370° C., or as high as about 380° C., about 390° C., about 400° C., about 450° C., about 500° C., about 550° C., about 600° C., or within any range encompassed by two of the foregoing values as endpoints, for example, from about 200° C. to about 500° C., or from about 250° C. to about 450° C., or from about 250° C. to about 400° C. As part of the catalyst activation, the catalyst may be exposed to an inert gas such as N₂. The pretreatment process may take as low as about 1 hour, about 2 hours, about 3 hours, or as high about 4 hours, about 5 hours, about 6 hours, about 10 hours, about 20 hours, or within any range encompassed by two of the foregoing values as endpoints, for example, from about 1 hours to about 20 hours, or from about 2 hours to about 10 hours, or from about 2 hours to about 4 hours.

When stainless steel is used, the catalyst may be pretreated by drying at a temperature of from about 200° C. to about 500° C., preferably from about 250° C. to about 450° C., most preferably from about 300° C. to about 400° C. under a flow of N₂ at 20 ml/min.

When fluorinated aluminum oxide is used, the catalyst may be pretreated by drying at a temperature of from about 200° C. to about 500° C., preferably from about 250° C. to about 450° C., most preferably from about 300° C. to about 400° C. under a flow of N₂ at 20 ml/min. The fluorinated aluminum oxide catalyst may be pretreated for about 3 hours, or about 4 hours, or about 5 hours, or within any range encompassed by two of the foregoing values as endpoints.

When fluorinated chromium oxide is used, the catalyst may be pretreated by slowly heating to a temperature of from about 200° C. to about 350° C., from about 250° C. to about 300° C., or up to about 280° C. under a flow of air at 100 ml/min, followed by drying at a temperature of from about 200° C. to about 500° C., preferably from about 250° C. to about 450° C., most preferably from about 300° C. to about 400° C. for about 3 hours, or about 4 hours, or about 5 hours before use.

When nickel alloy or a nickel-chromium alloy is used, the catalyst may be pretreated by drying at a temperature of from about 200° C. to about 500° C., preferably from about 250° C. to about 450° C., most preferably from about 300° C. to about 400° C. under a flow of N₂ at 20 ml/min. The fluorinated aluminum oxide catalyst may be pretreated for about 3 hours, or about 4 hours, or about 5 hours, or within any range encompassed by two of the foregoing values as endpoints.

### Process 1 Reaction Conditions (Tubular Reactor)

The temperature range for Process 1 in a tubular reactor may be as low as about 300° C., about 350° C., about 400° C., about 450° C., or as high as about 500° C., about 550° C., about 600° C., about 650° C., about 700° C., about 750° C., about 800° C., or within any range encompassed by two of the foregoing values as endpoints, for example, from about 300° C. to about 800° C., or preferably from about 300° C. to about 650° C., or more preferably from about 350° C. to about 600° C. Specific examples of additional suitable ranges are set forth below in Table 1.

TABLE 1
Reaction Temperature for Process 1 in a Tubular Reactor

| From (° C.) | To (° C.) |
|---|---|
| 300 | 800 |
| 300 | 750 |
| 300 | 700 |
| 300 | 650 |
| 300 | 600 |
| 350 | 800 |
| 350 | 750 |
| 350 | 700 |
| 350 | 650 |
| 350 | 600 |
| 300 | 350 |
| 350 | 400 |
| 400 | 450 |
| 450 | 500 |
| 500 | 550 |
| 550 | 600 |
| 600 | 650 |
| 650 | 700 |
| 700 | 750 |
| 750 | 800 |

The reactor (e.g., reactor 102 in FIG. 1) may be preheated to a desired reaction temperature (e.g., 500°). The feed stream may flow through heat exchangers that vaporizes the feed stream to a temperature substantially similar to the desired reaction temperature (e.g., 500° C.).

The pressure may be as little as about 1 psig, about 2 psig, about 3 psig, about 4 psig or about 5 psig, about 10 psig, about 15 psig, about 20 psig, about 25 psig, about 30 psig, about 35 psig, about 40 psig, about 50 psig, or within any range encompassed by two of the foregoing values as endpoints, for example, from about 1 psig to about 50 psig, or preferably from about 5 psig to about 30 psig, or more preferably from about 10 psig to about 20 psig. The reactor (e.g., reactor 102 in FIG. 1) may be kept at a pressure of above 15 psig (or above 1 atm). Specific examples of additional suitable ranges are set forth below in Table 2.

TABLE 2
Reaction Pressure for Process 1 in a Tubular Reactor

| From (psig) | To (psig) |
|---|---|
| 1 | 50 |
| 1 | 40 |
| 1 | 35 |
| 1 | 30 |
| 1 | 25 |
| 1 | 20 |
| 5 | 50 |
| 5 | 40 |
| 5 | 35 |
| 5 | 30 |
| 5 | 25 |
| 5 | 20 |
| 1 | 5 |
| 5 | 10 |
| 10 | 15 |
| 15 | 20 |
| 20 | 25 |
| 25 | 30 |
| 30 | 35 |
| 35 | 40 |
| 40 | 45 |
| 45 | 50 |

The contact time of the reactants with the catalyst may be as little as about 0.1 second, about 0.5 second, about 1 second, about 5 seconds, about 10 seconds, about 15 seconds, about 20 seconds, or as long as about 25 seconds, about 30 seconds, about 40 seconds, about 50 seconds, about 60 seconds, about 120 seconds, or within any range encompassed by two of the foregoing values as endpoints, for example, from about 0.1 second to about 5 seconds, or from about 5 seconds to about 10 seconds. Specific examples of additional suitable ranges are set forth below in Table 3.

TABLE 3
Contact Time for Process 1 in a Tubular Reactor

| From (seconds) | To (seconds) |
|---|---|
| 0.1 | 120 |
| 0.1 | 100 |
| 0.1 | 80 |
| 0.1 | 60 |
| 1 | 120 |
| 1 | 100 |
| 1 | 80 |
| 1 | 60 |
| 0.1 | 1 |
| 1 | 5 |
| 5 | 10 |
| 10 | 20 |
| 20 | 30 |
| 30 | 40 |
| 40 | 50 |
| 50 | 60 |
| 60 | 80 |
| 80 | 100 |
| 100 | 120 |

The reaction may also be conducted substantially in the absence of water. For example, the amount of water present during the reaction may be less than 1 mol %, less than 0.5 mol %, or less than 0.05 mol % based on a total amount of the reactants in the reactor.

### Catalysts for Process 1 (Autoclave Reactor)

No catalyst may be needed when using an autoclave reactor for carrying out Process 1. The autoclave reactor itself may include one or more tubes having inner surfaces contacting the feed stream that improves the isomerization reaction. The longer holding time by using an autoclave reactor may further improve the isomerization reaction.

Alternatively, solid catalyst including for example, iodine crystals, may be used for an autoclave process. The iodine crystals do not require pre-treatment or activation prior to the reaction. The iodine crystals may be preloaded into the autoclave reactor (e.g., reactor 102) before the pressure check.

### Process 1 Reaction Conditions (Autoclave Reactor)

The temperature range for the isomerization reaction may be as low as about 50° C., about 100° C., about 150° C., about 200° C., or as high as about 250° C., about 300° C., about 350° C., about 380° C., about 400° C., about 450° C., or within any range encompassed by two of the foregoing values as endpoints. The temperature may be preferably from about 100° C. to about 380° C., and more preferably from about 300° C. to about 350° C. Specific examples of additional suitable ranges are set forth below in Table 4.

TABLE 4
Reaction Temperature for Process 1 in an Autoclave Reactor

| From (° C.) | To (° C.) |
|---|---|
| 50 | 450 |
| 50 | 400 |
| 50 | 380 |
| 50 | 350 |
| 50 | 300 |
| 50 | 250 |
| 100 | 450 |
| 100 | 400 |
| 100 | 380 |
| 100 | 350 |
| 100 | 300 |
| 100 | 250 |
| 50 | 100 |
| 100 | 200 |
| 200 | 300 |
| 300 | 350 |
| 350 | 380 |
| 380 | 400 |
| 400 | 450 |

The reaction may be conducted at atmospheric pressure, super-atmospheric pressure or under vacuum. The super-atmospheric pressure may be from about 60 psig to about 120 psig, or from about 65 psig to about 100 psig, or from about 68 psig to about 78 psig. Specific examples of additional suitable ranges are set forth below in Table 5.

TABLE 5
Reaction Pressure for Process 1 in an Autoclave Reactor

| From (psig) | To (psig) |
|---|---|
| 60 | 120 |
| 60 | 110 |
| 60 | 100 |
| 60 | 90 |
| 60 | 80 |
| 60 | 78 |
| 65 | 120 |
| 65 | 110 |
| 65 | 100 |
| 65 | 90 |
| 65 | 80 |
| 65 | 78 |
| 60 | 65 |
| 65 | 68 |
| 68 | 70 |
| 70 | 75 |
| 75 | 78 |
| 78 | 80 |
| 80 | 90 |
| 90 | 100 |
| 100 | 110 |
| 110 | 120 |

Contact time of the reactants with the catalyst may range from about 0.1 hour to about 1 hour, or from about 0.2 hour to about 0.9 hour, or from about 0.3 hour to about 0.8 hour, or preferably from about 0.4 hour to about 0.6 hour, or more preferably about 0.5 hour, however, longer or shorter times can be used. Specific examples of additional suitable ranges are set forth below in Table 6.

TABLE 6
Contact Time for Process 1 in an Autoclave Reactor

| From (hour) | To (hour) |
|---|---|
| 0.1 | 1 |
| 0.1 | 0.9 |
| 0.1 | 0.8 |
| 0.1 | 0.7 |
| 0.1 | 0.6 |
| 0.2 | 1 |
| 0.2 | 0.9 |
| 0.2 | 0.8 |
| 0.2 | 0.7 |
| 0.2 | 0.6 |
| 0.3 | 1 |
| 0.3 | 0.9 |
| 0.3 | 0.8 |
| 0.3 | 0.7 |
| 0.3 | 0.6 |
| 0.4 | 1 |
| 0.4 | 0.9 |
| 0.4 | 0.8 |
| 0.4 | 0.7 |
| 0.4 | 0.6 |

The reaction may also be conducted in an inert atmosphere substantially in the absence of oxygen. For example, the amount of oxygen present during the reaction may be less than 10 mol. %, less than 5 mol. %, or less than 1 mol. % based on a total amount of the reactants in the reactor.

The reaction may also be conducted substantially in the absence of water. For example, the amount of water present during the reaction may be less than 1 mol. %, less than 0.5 mol. %, or less than 0.05 mol. % based on a total amount of the reactants in the reactor.

The autoclave process may include a non-catalytic isomerization reaction conducted without irradiation, for example, ultraviolet radiation having a wavelength of from about 100 nm to about 400 nm, electromagnetic radiation having a wavelength of from about 380 nm to about 760 nm, infrared radiation having a wavelength of from about 780 nm to about 1 mm, and/or near-infrared radiation having a wavelength of from about 750 nm to about 2500 nm.

### Process 1 Products

As demonstrated by the Examples herein, Process 1, using either a catalytic or a non-catalytic process, may achieve a selectivity to the HFO-1132(E) product of greater than about 20%, greater than about 30%, greater than about 40%, greater than about 50%, greater than about 60%, greater than about 70%, greater than about 80%, greater than about 85%, greater than about 90%, greater than about 95%, greater than about 98%, greater than about 99%, greater than about 99.9%, greater than about 99.99%, and for each of the foregoing, less than or equal to 100%, or within any range encompassed by two of the foregoing values as endpoints, based on total amount of organic components of the product composition. Specific examples of additional suitable ranges are set forth below in Table 7.

TABLE 7
Selectivity to HFO-1132(E) in Process 1

| From (%) | To (%) |
|---|---|
| 20 | ≤100 |
| 30 | ≤100 |
| 40 | ≤100 |
| 50 | ≤100 |
| 60 | ≤100 |
| 70 | ≤100 |
| 80 | ≤100 |
| 85 | ≤100 |
| 90 | ≤100 |
| 95 | ≤100 |
| 98 | ≤100 |
| 99 | ≤100 |
| 99.9 | ≤100 |
| 99.99 | ≤100 |

As also demonstrated by the Examples herein, Process 1, using either a catalytic or a non-catalytic reaction in a once-through process, may achieve a conversion of HFO-1132(Z) to HFO-1132(E) of greater than about 4%, greater than about 7%, greater than about 10%, greater than about 15%, greater than about 20%, greater than about 25%, greater than about 30%, greater than about 35%, greater than about 40%, and for each of the foregoing, less than or equal to 50%, or within any range encompassed by two of the foregoing values as endpoints, for example, from about 5% to about 50%, or from about 30% to about 50%, based on total amount of organic components of the product composition. Specific examples of additional suitable ranges are set forth below in Table 8.

TABLE 8
Conversion of HFO-1132(Z) to HFO-1132(E) in Process 1

| From (%) | To (%) |
|---|---|
| 4 | 50 |
| 7 | 50 |
| 10 | 50 |
| 13 | 50 |
| 15 | 50 |
| 18 | 50 |
| 20 | 50 |
| 23 | 50 |
| 25 | 50 |
| 28 | 50 |
| 30 | 50 |
| 32 | 50 |
| 35 | 50 |
| 40 | 50 |

The end product of an integrated process (e.g., after distillation and/or purification) may include a mixture of HFO-1132(Z) and HFO-1132(E), for example, from about 80% to about 99.99% of HFO-1132(E) and from about 0.01% to about 20% of 1132(Z), or from about 85% to about 99.99% of 1132(E) and from about 0.01% to about 15% 1132(Z), or from about 90% to about 99.99% of 1132(E) and from about 0.01% to about 10% 1132(Z), or from about 95% to about 99.99% of 1132(E) and from about 0.01% to about 5% 1132(Z), or from about 98% to about 99.99% of 1132(E) and from about 0.01% to about 2% 1132(Z), or within any range encompassing any two of these values as endpoints, based on total amount of organic components of the end product.

For the catalytic process, it may also be advantageous to periodically regenerate the catalyst after prolonged use while in place in the tubular reactor. Regeneration of the catalyst may be accomplished by any means known in the art, for example, by passing air or air diluted with nitrogen over the catalyst at temperatures of from about 100° C. to about 400° C., preferably from about 200° C. to about 375° C., for from about 0.5 hour to about 3 days. This may be followed by hydrofluorination treatment at temperatures of from about 100° C. to about 400° C., or from about 200° C. to about 350° C.

Process 1 may be conducted in a tubular reactor using stainless steel as a catalyst, at a temperature of from about 500° C. to about 700° C., at a pressure of from about 10 psig to about 20 psig, and with a contact time of from about 0.5 second to about 3 seconds. The amount of 1132(E) in the product composition after a single pass reaction may be at least about 32% and less than or equal to 50%, based on the total amount of organic components of the product composition. The amount of 1132(E) in the end product in an integrated process may be at least about 99.9% and less than or equal to 100%, based on total amount of organic components of the end product.

Process 1 may be conducted in a tubular reactor using a catalyst including about 75% to about 78% nickel, about 20% to about 23% chromium, about 1.5% of manganese, and a balance of iron, at a temperature of from about 450° C. to about 650° C., at a pressure of from about 3 psig to about 10 psig, and with a contact time of from about 0.5 second to about 2 seconds. The amount of 1132(E) in the product mixture after a single pass reaction may be at least about 29% and less than or equal to 50%, based on the total amount of organic components of the product composition. The amount of 1132(E) in the end product mixture in an integrated process may be at least about 99.9% and less than or equal to 100%, based on total amount of organic components of the end product.

Process 1 may be conducted in a tubular reactor using a catalyst including about 99.6% nickel, at a temperature of from about 450° C. to about 650° C., and at a pressure of from about 3 psig to about 32 psig, and with a contact time of from about 0.5 second to about 2 seconds. The amount of 1132(E) in the product mixture after a single pass reaction may be at least about 26% and less than or equal to 50%, based on the total amount of organic components of the product composition. The amount of 1132(E) in the end product mixture in an integrated process may be at least about 99.9% and less than or equal to 100%, based on total amount of organic components of the end product.

Process 1 may be conducted in an autoclave reactor lined with iodine crystals, at a temperature of from about 300° C. to about 380° C., at a pressure of from about 68 psig to about 78 psig, and with a contact time of about 0.5 hour. The amount of 1132(E) in the product mixture after a single pass reaction may be at least about 36% and less than or equal to 50%, based on the total amount of organic components of the product composition. The amount of 1132(E) in the end product mixture in an integrated process may be at least about 99.9% and less than or equal to 100%, based on total amount of organic components of the end product.

### IV. Process 2

### General Process 2

As discussed above, a feed stream may include 1,2-difluoroethylene (HFO-1132) obtained via various methods or starting materials and having a mixture containing both the trans-HFO-1132(E) and cis-HFO-1132(Z) isomers, or substantially pure HFO-1132(E).

The trans-1,2-difluoroethylene HFO-1132(E) isomer may be converted to the cis-1,2-difluoroethylene (HFO-1132(Z)) isomer either by exposure to heat and/or a catalyst and further purified to form an end product comprising, consisting essentially of, or consisting of, the cis-1,2-difluoroethylene (HFO-1132(Z)) isomer in high purity, such as at least about 95 wt. %, at least about 99.0 wt. %, at least about 99.9 wt. %, at least about 99.99 wt. % or greater, and for each of the foregoing, less than or equal to 100%, based on total amount of organic components of the end product composition.

The isomerization reaction may be conducted in a suitable reaction vessel or reactor. The reactor is preferably constructed from materials which are resistant to corrosion such as nickel and its alloys, including Hastelloy (for example, Hastelloy C276), Inconel (for example Inconel 600), and Monel. The reactor may also be a stainless steel reactor. In addition, the reactor may be made to include materials that may serve a similar function as a catalyst, but for a non-catalytic reaction.

The reactor may be first cleaned and flushed with an inert gas such as nitrogen. For catalytic processes, the reactor may then be packed with a catalyst such as those described below. The catalyst may be pretreated within the reactor such as by drying in the manner described further below, followed by metering the reactants into the reactor to initiate the reaction. The process flow may be in the down or up direction through a bed of the catalyst. Products may be flowed through one or more scrubbers to remove byproducts contained in the feed stream from the stream, such as hydrogen fluoride (HF) and/or hydrogen chloride (HCl), and the reaction products may be collected by capture in a cooled cylinder, for example.

### Process Flow (Process 2)

Process 2 may be carried out according to a process flow substantially similar as described above and with reference to the schematic process flow diagram as shown in FIG. 1. Process flow may be carried out in a tubular reactor or an autoclave reactor.

For a tubular reactor, the flow rate may be from about 1 g/hr to about 30 g/hr, or from about 1.2 g/hr to about 25 g/hr, or from about 5 g/hr to about 15 g/hr. The contact time may be from about 0.1 second to about 15 seconds, or from about 0.5 second to about 11 seconds, or from about 1 second to about 10 seconds. The process through a tubular reactor may be a continuous flowing process with relatively short contact time.

For an autoclave reactor, the contact time may be from about 0.1 hour to about 3 hours, or from about from about 0.5 hour to about 2.5 hours, or from about 1 hour to about 2 hours. The process through an autoclave reactor may be a batch like process (e.g., a semi-batch process) with longer holding time compared to a tubular reactor.

### Catalysts for Process 2 (Tubular Reactor)

Suitable catalysts for using with a tubular reactor in Process 2 may include stainless steel (e.g., 316 stainless steel Pro-Pak®), metal oxides (e.g., chromium oxide, aluminum oxide), or metal alloys such as nickel alloy (e.g., Nickel Alloy 200 Pro-Pak®). Fluorination treatment of the catalyst may be conducted using anhydrous HF under conditions effective to convert a portion of metal oxides into corresponding metal fluorides, such as via the procedure disclosed in U.S. Pat. No. 6,780,815 to Cerri et al., the disclosure of which is expressly incorporated by reference herein. Other suitable catalysts for the catalytic reaction include metal fluorides such as chromium fluoride, alumina fluoride, iron fluoride, magnesium fluoride, and various combinations of thereof.

The catalyst used in Process 2 with a tubular reactor may have a proper BET (Brunauer, Emmet, and Teller) surface area. The BET surface area of the catalyst may be as low as about 10 m²/g, about 20 m²/g, about 30 m²/g, about 40 m²/g, about 50 m²/g, about 60 m²/g, about 70 m²/g, about 80 m²/g, about 90 m²/g, about 100 m²/g, or as high as about 110 m²/g, about 120 m²/g, about 130 m²/g, about 140 m²/g, about 150 m²/g, about 175 m²/g, about 200 m²/g, about 225 m²/g, about 250 m²/g, about 300 m²/g, or within any range encompassed by any of the foregoing values as endpoints, for example, from about 10 m²/g to about 300 m²/g, or from about 20 m²/g to about 250 m²/g.

For metal oxides catalysts, the BET surface area may be preferably greater than about 100 m²/g. For fluorinated metal oxides catalysts, the BET surface area may be preferably greater than about 20 m²/g. The BET analysis is the standard method for determining surface areas from nitrogen adsorption isotherms. The BET surface areas of catalysts may be measured using TriStar II Micromeritics instrument. Catalyst samples are degassed before the analysis using FlowPrep 060 instrument.

### Catalyst Pretreatment for Process 2 (Tubular Reactor)

The catalyst may be pretreated by drying at elevated temperatures, as low as about 200° C., about 250° C., about 300° C., about 350° C., about 360° C., about 370° C., or as high as about 380° C., about 390° C., about 400° C., about 450° C., about 500° C., about 550° C., about 600° C., or within any range encompassed by two of the foregoing values as endpoints. As part of the catalyst activation, the catalyst may be exposed to an inert gas such as N₂. The pretreatment process may take as low as about 1 hour, about 2 hours, about 3 hours, or as high about 4 hours, about 5 hours, about 6 hours, about 10 hours, about 20 hours, or within any range encompassed by two of the foregoing values as endpoints such as about 2 hours to about 4 hours, for example.

When stainless steel is used, the catalyst may be pretreated by drying at a temperature of from about 200° C. to about 500° C., preferably from about 250° C. to about 450° C., most preferably from about 300° C. to about 400° C. under a flow of N₂ at 20 ml/min.

When fluorinated aluminum oxide is used, the catalyst may be pretreated by drying at a temperature of from about 200° C. to about 500° C., preferably from about 250° C. to about 450° C., most preferably from about 300° C. to about 400° C. under a flow of N₂ at 20 ml/min. The fluorinated aluminum oxide catalyst may be pretreated for about 3 hours, or about 4 hours, or about 5 hours, or within any range encompassed by two of the foregoing values as endpoints.

When fluorinated chromium oxide is used, the catalyst may be pretreated by slowly heating to a temperature of from about 200° C. to about 350° C., from about 250° C. to about 300° C., or from about 250° C. to about 280° C. under a flow of air at 100 ml/min, followed by drying at a temperature of from about 200° C. to about 500° C., preferably from about 250° C. to about 450° C., most preferably from about 300° C. to about 400° C. for about 3 hours, or about 4 hours, or about 5 hours before use.

When nickel alloy is used, the catalyst may be pretreated by drying at a temperature of from about 200° C. to about 500° C., preferably from about 250° C. to about 450° C., most preferably from about 300° C. to about 400° C. under a flow of N₂ at 20 ml/min. The fluorinated aluminum oxide catalyst may be pretreated for about 3 hours, or about 4 hours, or about 5 hours, or within any range encompassed by two of the foregoing values as endpoints.

### Reaction Conditions for Process 2 (Tubular Reactor)

The temperature range for Process 1 in a tubular reactor may be as low as about 300° C., about 350° C., about 400° C., about 450° C., or as high as about 500° C., about 550° C., about 600° C., about 650° C., about 700° C., about 750° C., about 800° C., about 850° C., or within any range encompassed by two of the foregoing values as endpoints, for example, from about 300° C. to about 750° C., or preferably from about 350° C. to about 700° C. Specific examples of additional suitable ranges are set forth below in Table 9.

TABLE 9
Reaction Temperature for Process 2 in a Tubular Reactor

| From (° C.) | To (° C.) |
|---|---|
| 300 | 850 |
| 300 | 800 |
| 300 | 750 |
| 300 | 700 |
| 300 | 650 |
| 300 | 600 |
| 300 | 550 |
| 300 | 500 |
| 350 | 850 |
| 350 | 800 |
| 350 | 750 |
| 350 | 700 |
| 350 | 650 |
| 350 | 600 |
| 350 | 550 |
| 300 | 350 |
| 350 | 400 |
| 400 | 450 |
| 450 | 500 |
| 500 | 550 |
| 550 | 600 |
| 600 | 650 |
| 650 | 700 |
| 700 | 750 |
| 750 | 800 |
| 800 | 850 |

The pressure may be as little as about 1 psig, about 2 psig, about 3 psig, about 4 psig or about 5 psig, about 10 psig, about 15 psig, about 20 psig, about 25 psig, or as high as about 30 psig, about 35 psig, about 40 psig, about 50 psig, about 55 psig, about 60 psig, about 65 psig, about 70 psig, or within any range encompassed by two of the foregoing values as endpoints, for example, from about 1 psig to about 60 psig, or from about 5 psig to about 55 psig, or preferably from about 10 psig to about 50 psig, or more preferably from about 15 psig to about 45 psig. Specific examples of additional suitable ranges are set forth below in Table 10.

TABLE 10
Reaction Pressure for Process 2 in a Tubular Reactor

| From (psig) | To (psig) |
|---|---|
| 1 | 70 |
| 1 | 60 |
| 1 | 55 |
| 1 | 50 |
| 1 | 45 |
| 1 | 40 |
| 1 | 35 |
| 1 | 30 |
| 1 | 25 |
| 1 | 20 |
| 5 | 70 |
| 5 | 60 |
| 5 | 55 |
| 5 | 50 |
| 5 | 45 |
| 5 | 40 |
| 1 | 5 |
| 5 | 10 |
| 10 | 15 |
| 15 | 20 |
| 20 | 25 |
| 25 | 30 |
| 30 | 35 |
| 35 | 40 |
| 40 | 45 |
| 45 | 50 |
| 50 | 55 |
| 55 | 60 |
| 60 | 65 |
| 65 | 70 |

The contact time of the reactants with the catalyst may be as little as about 0.1 second, about 0.5 second, about 1 second, about 5 seconds, about 10 seconds, about 15 seconds or about 20 seconds, or as long as about 25 seconds, about 30 seconds, about 40 seconds, about 50 seconds, about 60 seconds, about 120 seconds, about or within any range encompassed by two of the foregoing values as endpoints. For example, the contact time may be from about 0.5 second to about 5 seconds, or from about 5 seconds to about 11 seconds. Specific examples of additional suitable ranges are set forth below in Table 11.

TABLE 11
Contact Time for Process 2 in a Tubular Reactor

| From (seconds) | To (seconds) |
|---|---|
| 0.1 | 120 |
| 0.1 | 100 |
| 0.1 | 80 |
| 0.1 | 60 |
| 1 | 120 |
| 1 | 100 |
| 1 | 80 |
| 1 | 60 |
| 0.1 | 0.5 |
| 0.5 | 1 |
| 1 | 5 |
| 5 | 11 |
| 11 | 15 |
| 15 | 20 |
| 20 | 30 |
| 30 | 40 |
| 40 | 50 |
| 50 | 60 |
| 60 | 80 |
| 80 | 100 |
| 100 | 120 |

The reaction may also be conducted substantially in the absence of water. For example, the amount of water present during the reaction may be less than 1 mol %, less than 0.5 mol %, or less than 0.05 mol %, and for each of the foregoing, equal to or more than 0 mol %, based on a total amount of the reactants in the reactor.

### Catalysts for Process 2 (Autoclave Reactor)

No catalyst may be needed when using an autoclave reactor for carrying out Process 1. The autoclave reactor itself may include one or more tubes having inner surfaces contacting the feed stream that improves the isomerization reaction. The longer holding time by using an autoclave reactor may further improve the isomerization reaction.

Alternatively, solid catalyst including for example, iodine crystals, may be used for an autoclave process. The iodine crystals do not require pre-treatment or activation prior to the reaction. The iodine crystals may be preloaded into the autoclave reactor (e.g., reactor 102) before the pressure check.

### Reaction Conditions (Autoclave Reactor)

The temperature range for the isomerization reaction may be as low as about 50° F., about 100° C., about 150° C., about 200° C., or as high as about 250°), about 300° C., about 350° C., about 400° C., about 450° C., or within any range encompassed by two of the foregoing values as endpoints, for example, preferably from about 100° C. to about 350° C., or more preferably from about 250° C. to about 350° C. Specific examples of additional suitable ranges are set forth below in Table 12.

TABLE 12
Reaction Temperature for Process 2 in an Autoclave Reactor

| From (° C.) | To (° C.) |
|---|---|
| 50 | 450 |
| 50 | 400 |
| 50 | 380 |
| 50 | 350 |
| 50 | 300 |
| 50 | 250 |
| 100 | 450 |
| 100 | 400 |
| 100 | 380 |
| 100 | 350 |
| 100 | 300 |
| 100 | 250 |
| 50 | 100 |
| 100 | 200 |
| 200 | 250 |
| 250 | 350 |
| 350 | 380 |
| 380 | 400 |
| 400 | 450 |

The reaction may be conducted at atmospheric pressure, super-atmospheric pressure or under vacuum. The reaction pressure may be as low as about 50 psig, 60 psig, 65 psig, 70 psig, 100 psig, 150 psig, 155 psig, or as high as about 300 psig, about 320 psig, about 350 psig, about 400 psig, or within any range encompassed by two of the foregoing values as endpoints, for example, from about 55 psig to about 400 psig, or from about 100 psig to about 350 psig. Specific examples of additional suitable ranges are set forth below in Table 13.

TABLE 13
Reaction Pressure for Process 1 in an Autoclave Reactor

| From (psig) | To (psig) |
|---|---|
| 50 | 400 |
| 50 | 350 |
| 50 | 300 |
| 50 | 200 |
| 50 | 150 |
| 50 | 100 |
| 65 | 400 |
| 65 | 350 |
| 65 | 300 |
| 65 | 200 |
| 65 | 150 |
| 65 | 60 |
| 60 | 65 |
| 65 | 70 |
| 70 | 100 |
| 100 | 150 |
| 150 | 155 |
| 155 | 200 |
| 200 | 250 |
| 250 | 300 |
| 300 | 320 |
| 320 | 340 |
| 340 | 350 |
| 350 | 400 |

Contact time of the reactants with the catalyst and/or the reactor in a non-catalytic reaction may be as low as about 0.1 hour, 0.2 hour, 0.3 hour, 0.5 hour, 0.8 hour, or as high as 1 hour, 1.5 hour, 2.0 hour, 2.5 hour, or within any range encompassed by two of the foregoing values as endpoints, for example, from about 0.1 hour to about 2.5 hours, however, longer or shorter times can be used. Specific examples of additional suitable ranges are set forth below in Table 14.

TABLE 14
Contact Time for Process 2 in an Autoclave Reactor

| From (hour) | To (hour) |
|---|---|
| 0.1 | 2.5 |
| 0.1 | 2.0 |
| 0.1 | 1.5 |
| 0.1 | 1 |
| 0.1 | 0.5 |
| 0.2 | 2.5 |
| 0.2 | 2.0 |
| 0.2 | 1.5 |
| 0.2 | 1 |
| 0.2 | 0.5 |
| 0.3 | 2.5 |
| 0.3 | 2.0 |
| 0.3 | 1.5 |
| 0.3 | 1 |
| 0.3 | 0.5 |
| 0.1 | 0.2 |
| 0.2 | 0.3 |
| 0.3 | 0.5 |
| 0.5 | 1 |
| 1 | 1.5 |
| 1.5 | 2 |
| 2 | 2.5 |

### Process 2 Products

As demonstrated by the Examples herein, Process 2, using either a catalytic or a non-catalytic process, may achieve a selectivity to the HFO-1132(Z) product of greater than about 20%, greater than about 30%, greater than about 40%, greater than about 50%, greater than about 60%, greater than about 70%, greater than about 80%, greater than about 85%, greater than about 90%, greater than about 95%, greater than about 98%, greater than about 99%, greater than about 99.9%, greater than about 99.99%, and for each of the foregoing, less than or equal to 100%, or within any range encompassed by two of the foregoing values as endpoints, based on total amount of organic components of the product composition. Specific examples of additional suitable ranges are set forth below in Table 15.

TABLE 15
Selectivity to HFO-1132(Z) in Process 2

| From (%) | To (%) |
|---|---|
| 20 | ≤100 |
| 30 | ≤100 |
| 40 | ≤100 |
| 50 | ≤100 |
| 60 | ≤100 |
| 70 | ≤100 |
| 80 | ≤100 |
| 85 | ≤100 |
| 90 | ≤100 |
| 95 | ≤100 |
| 98 | ≤100 |
| 99 | ≤100 |
| 99.9 | ≤100 |
| 99.99 | ≤100 |

As also demonstrated by the Examples herein, Process 2, using either a catalytic or a non-catalytic reaction in a once-through process, may achieve a conversion of HFO-1132(E) to HFO-1132(Z) of greater than about 4%, greater than about 5%, greater than about 8%, greater than about 10%, greater than about 15%, greater than about 20%, greater than about 25%, greater than about 30%, greater than about 35%, greater than about 40%, greater than about 45%, greater than about 50%, greater than about 55%, greater than about 60%, greater than about 65%, greater than about 69%, and for each of the foregoing, less than or equal to 80%, or within any range encompassed by two of the foregoing values as endpoints, for example, from about 10% to about 100%, or from about 20% to about 100%, based on total amount of organic components of the product composition. Specific examples of additional suitable ranges are set forth below in Table 16.

TABLE 16
Conversion of HFO-1132(E) to HFO-1132(Z) in Process 2

| From (%) | To (%) |
|---|---|
| 5 | 80 |
| 10 | 80 |
| 15 | 80 |
| 20 | 80 |
| 25 | 80 |
| 30 | 80 |
| 35 | 80 |
| 40 | 80 |
| 45 | 80 |
| 50 | 80 |
| 55 | 80 |
| 60 | 80 |
| 65 | 80 |
| 69 | 80 |

The end product in an integrated process may include a mixture of HFO-1132(Z) and HFO-1132(E), for example, from about 80% to about 99.99% of HFO-1132(Z) and from about 0.01% to about 20% of 1132(E), or from about 85% to about 99.99% of 1132(Z) and from about 0.01% to about 15% 1132(E), or from about 90% to about 99.99% of 1132(Z) and from about 0.01% to about 10% 1132(E), or from about 95% to about 99.99% of 1132(Z) and from about 0.01% to about 5% 1132(E), or from about 98% to about 99.99% of 1132(Z) and from about 0.01% to about 2% 1132(E), or within any range encompassing any two of these values as endpoints, based on the total amount of organic components of the end product.

For the catalytic process, it may also be advantageous to periodically regenerate the catalyst after prolonged use while in place in the tubular reactor. Regeneration of the catalyst may be accomplished by any means known in the art, for example, by passing air or air diluted with nitrogen over the catalyst at temperatures of from about 100° C. to about 400° C., preferably from about 200° C. to about 375° C., for from about 0.5 hour to about 3 days. This may be followed by hydrofluorination treatment at temperatures of from about 100° C. to about 400° C., or from about 200° C. to about 350° C.

Process 2 may be conducted in a tubular reactor using stainless steel as a catalyst, at a temperature of from about 500° C. to about 700° C., at a pressure of from about 10 psig to about 20 psig, and with a contact time of from about 1 second to about 5 seconds. The amount of 1132(E) in the product mixture after a single pass reaction may be at least about 24% and less than or equal to 80%, based on the total amount of organic components of the product composition. The amount of 1132(E) in the end product mixture in an integrated process may be at least about 99.9% and less than or equal to 100%, based on the total amount of organic components of the end product.

Process 2 may be conducted in a tubular reactor using a catalyst including fluorinated aluminum oxide, at a temperature of from about 350° C. to about 450° C., at a pressure of from about 15 psig to about 45 psig, and with a contact time of from about 3 seconds to about 10 seconds. The amount of 1132(E) in the product mixture after a single pass reaction may be at least about 42% and less than or equal to 80%, based on the total amount of organic components of the product composition. The amount of 1132(E) in the end product mixture in an integrated process may be at least about 99.9% and less than or equal to 100%, based on the total amount of organic components of the end product.

Process 2 may be conducted in a tubular reactor using a catalyst including fluorinated chromium oxide, at a temperature of about 350° C. to about 450° C., at a pressure of from about 10 psig to about 20 psig, and with a contact time of from about 2 seconds to about 4 seconds. The amount of 1132(E) in the product mixture after a single pass reaction may be at least about 27% and less than or equal to 80%, based on the total amount of organic components of the product composition. The amount of 1132(E) in the end product mixture in an integrated process may be at least about 99.9% and less than or equal to 100%, based on the total amount of organic components of the end product.

Process 2 may be conducted in an autoclave reactor lined with iodine crystals, at a temperature of from about 250° C. to about 350° C., and with a contact time of from about 0.5 hour to about 2.5 hours. The amount of 1132(E) in the product mixture after a single pass reaction may be at least about 57% and less than or equal to 80%, based on the total amount of organic components of the product composition. The amount of 1132(E) in the end product mixture in an integrated process may be at least about 99.9% and less than or equal to 100%, based on the total amount of organic components of the end product.

A summary of the catalyst used and reaction conditions for Process 1 in a tubular reactor as discussed above are summarized in Table 17 below.

TABLE 17
Catalyst and Reaction Conditions for
Process 1 in a Tubular Reactor

| | Temperature | Pressure | Contact |
|---|---|---|---|
| Catalyst | (° C.) | (psig) | Time (sec) |
| Stainless steel | 300-800 | 1-50 | 0.1-10 |
| Stainless steel | 500-600 | 1-50 | 0.1-10 |
| Stainless steel | 300-800 | 10-20 | 0.1-10 |
| Stainless steel | 500-600 | 10-20 | 0.1-10 |
| Stainless steel | 300-800 | 1-50 | 0.1-5 |
| Stainless steel | 500-600 | 1-50 | 0.1-5 |
| Stainless steel | 300-800 | 10-20 | 0.1-5 |
| Stainless steel | 500-600 | 10-20 | 0.1-5 |
| Fluorinated aluminum oxide | 300-800 | 1-50 | 0.1-10 |
| Fluorinated aluminum oxide | 350-650 | 1-50 | 0.1-10 |
| Fluorinated aluminum oxide | 300-800 | 10-20 | 0.1-10 |
| Fluorinated aluminum oxide | 350-650 | 10-20 | 0.1-10 |
| Fluorinated aluminum oxide | 300-800 | 1-50 | 0.5-5 |
| Fluorinated aluminum oxide | 350-650 | 1-50 | 0.5-5 |
| Fluorinated aluminum oxide | 300-800 | 10-20 | 0.5-5 |
| Fluorinated aluminum oxide | 350-650 | 10-20 | 0.5-5 |
| Fluorinated chromium oxide | 300-800 | 1-50 | 0.1-10 |
| Fluorinated chromium oxide | 350-650 | 1-50 | 0.1-10 |
| Fluorinated chromium oxide | 300-800 | 10-20 | 0.1-10 |
| Fluorinated chromium oxide | 350-650 | 10-20 | 0.1-10 |
| Fluorinated chromium oxide | 300-800 | 1-50 | 0.5-5 |
| Fluorinated chromium oxide | 350-650 | 1-50 | 0.5-5 |
| Fluorinated chromium oxide | 300-800 | 10-20 | 0.5-5 |
| Fluorinated chromium oxide | 350-650 | 10-20 | 0.5-5 |
| Nickel | 300-800 | 1-50 | 0.1-10 |
| Nickel | 450-550 | 1-50 | 0.1-10 |
| Nickel | 300-800 | 10-20 | 0.1-10 |
| Nickel | 450-550 | 10-20 | 0.1-10 |
| Nickel | 300-800 | 1-50 | 0.5-5 |
| Nickel | 450-550 | 1-50 | 0.5-5 |
| Nickel | 300-800 | 10-20 | 0.5-5 |
| Nickel | 450-550 | 10-20 | 0.5-5 |
| Nickel alloy | 300-800 | 1-50 | 0.1-10 |
| Nickel alloy | 450-550 | 1-50 | 0.1-10 |
| Nickel alloy | 300-800 | 10-20 | 0.1-10 |
| Nickel alloy | 450-550 | 10-20 | 0.1-10 |
| Nickel alloy | 300-800 | 1-50 | 0.1-5 |
| Nickel alloy | 450-550 | 1-50 | 0.1-5 |
| Nickel alloy | 300-800 | 10-20 | 0.1-5 |
| Nickel alloy | 450-550 | 10-20 | 0.1-5 |

A summary of the catalyst used and reaction conditions for Process 1 in an autoclave reactor as discussed above are summarized in Table 18 below.

TABLE 18
Catalyst and Reaction Conditions for
Process 1 in an Autoclave Reactor

| | Temperature | Pressure | |
|---|---|---|---|
| Catalyst | (° C.) | (psig) | Contact Time (hour) |
| Iodine crystals | 50-450 | 60-120 | 0.1-1 |
| Iodine crystals | 100-380 | 60-120 | 0.1-1 |
| Iodine crystals | 50-450 | 65-100 | 0.1-1 |
| Iodine crystals | 100-380 | 65-100 | 0.1-1 |
| Iodine crystals | 50-450 | 60-120 | 0.4-0.6 |
| Iodine crystals | 100-380 | 60-120 | 0.4-0.6 |
| Iodine crystals | 50-450 | 65-100 | 0.4-0.6 |
| Iodine crystals | 100-380 | 65-100 | 0.4-0.6 |
| None | 50-450 | 60-120 | 0.1-1 |
| None | 100-380 | 60-120 | 0.1-1 |
| None | 50-450 | 65-100 | 0.1-1 |
| None | 100-380 | 65-100 | 0.1-1 |
| None | 50-450 | 60-120 | 0.4-0.6 |
| None | 100-380 | 60-120 | 0.4-0.6 |
| None | 50-450 | 65-100 | 0.4-0.6 |
| None | 100-380 | 65-100 | 0.4-0.6 |

A summary of the catalyst used and reaction conditions for Process 2 in a tubular reactor as discussed above are summarized in Table 19 below.

TABLE 19
Catalyst and Reaction Conditions for
Process 2 in a Tubular Reactor

| | Temperature | Pressure | Contact |
|---|---|---|---|
| Catalyst | (° C.) | (psig) | Time (sec) |
| Stainless steel | 300-750 | 1-60 | 0.1-120 |
| Stainless steel | 350-700 | 1-60 | 0.1-120 |
| Stainless steel | 300-750 | 15-45 | 0.1-120 |
| Stainless steel | 350-700 | 15-45 | 0.1-120 |
| Stainless steel | 300-750 | 1-60 | 0.5-20 |
| Stainless steel | 350-700 | 1-60 | 0.5-20 |
| Stainless steel | 300-750 | 15-45 | 0.5-20 |
| Stainless steel | 350-700 | 15-45 | 0.5-20 |
| Fluorinated aluminum oxide | 300-750 | 1-60 | 0.1-120 |
| Fluorinated aluminum oxide | 350-700 | 1-60 | 0.1-120 |
| Fluorinated aluminum oxide | 300-750 | 15-45 | 0.1-120 |
| Fluorinated aluminum oxide | 350-700 | 15-45 | 0.1-120 |
| Fluorinated aluminum oxide | 300-750 | 1-60 | 0.5-20 |
| Fluorinated aluminum oxide | 350-700 | 1-60 | 0.5-20 |
| Fluorinated aluminum oxide | 300-750 | 15-45 | 0.5-20 |
| Fluorinated aluminum oxide | 350-700 | 15-45 | 0.5-20 |
| Fluorinated chromium oxide | 300-750 | 1-60 | 0.1-120 |
| Fluorinated chromium oxide | 350-700 | 1-60 | 0.1-120 |
| Fluorinated chromium oxide | 300-750 | 15-45 | 0.1-120 |
| Fluorinated chromium oxide | 350-700 | 15-45 | 0.1-120 |
| Fluorinated chromium oxide | 300-750 | 1-60 | 0.5-20 |
| Fluorinated chromium oxide | 350-700 | 1-60 | 0.5-20 |
| Fluorinated chromium oxide | 300-750 | 15-45 | 0.5-20 |
| Fluorinated chromium oxide | 350-700 | 15-45 | 0.5-20 |
| Nickel alloy | 300-750 | 1-60 | 0.1-120 |
| Nickel alloy | 350-700 | 1-60 | 0.1-120 |
| Nickel alloy | 300-750 | 15-45 | 0.1-120 |
| Nickel alloy | 350-700 | 15-45 | 0.1-120 |
| Nickel alloy | 300-750 | 1-60 | 0.5-20 |
| Nickel alloy | 350-700 | 1-60 | 0.5-20 |
| Nickel alloy | 300-750 | 15-45 | 0.5-20 |
| Nickel alloy | 350-700 | 15-45 | 0.5-20 |

A summary of the catalyst used and reaction conditions for Process 2 in an autoclave reactor as discussed above are summarized in Table 20 below.

TABLE 20
Catalyst and Reaction Conditions for
Process 2 in an Autoclave Reactor

| | Temperature | Pressure | Contact |
|---|---|---|---|
| Catalyst | (° C.) | (psig) | Time (hour) |
| Iodine crystals | 100-400 | 50-400 | 0.1-3 |
| Iodine crystals | 250-350 | 50-400 | 0.1-3 |
| Iodine crystals | 100-400 | 100-350 | 0.1-3 |
| Iodine crystals | 250-350 | 100-350 | 0.1-3 |
| Iodine crystals | 100-400 | 50-400 | 0.5-2.5 |
| Iodine crystals | 250-350 | 50-400 | 0.5-2.5 |
| Iodine crystals | 100-400 | 100-350 | 0.5-2.5 |
| Iodine crystals | 250-350 | 100-350 | 0.5-2.5 |
| None | 100-400 | 50-400 | 0.1-3 |
| None | 250-350 | 50-400 | 0.1-3 |
| None | 100-400 | 100-350 | 0.1-3 |
| None | 250-350 | 100-350 | 0.1-3 |
| None | 100-400 | 50-400 | 0.5-2.5 |
| None | 250-350 | 50-400 | 0.5-2.5 |
| None | 100-400 | 100-350 | 0.5-2.5 |
| None | 250-350 | 100-350 | 0.5-2.5 |

### EXAMPLES

### Example 1

### Isomerization of 1132(Z) to 1132(E) (Single Pass Through a Tubular Reactor)

The process was carried out as described above and shown in FIG. 1 under conditions included in Table 21 below. The volume of the tubular reactor was 4.29 ml. For temperatures below 500° C., the feed stream (reactant composition) included 99.97% cis-1,2-difluoroethene (1132(Z)) and 0.02% trans-1,2-difluoroethylene (1132(E)). For temperature above 500° C., the feed stream (reactant composition) included 99.3% cis-1,2-difluoroethene (1132(Z)) and 0.5% trans-1,2-difluoroethylene (1132(E)). The reaction pressure was at 15 psig. The conversion rate of 1132(Z) to 1132(E) after a single pass through the reactor was from about 3.3% to about 37.69%, based on total amount of organic components of the product composition. The selectivity to desired product (1132(E)) was from about 98.88% to about 99.98%, based on total amount of organic components of the product composition. The reaction may be repeated in an integrated process to form an end product including an amount of 1132(E) of greater than about 90% and less than or equal to 100%, or greater than about 95% and less than or equal to 100%, based on total amount of organic components of the product composition.

TABLE 21
Process conditions and product compositions for a single pass through a
tubular reactor in Example 1

| T | Flow rate | | Catalyst | Product | Contact | Conversion |
|---|---|---|---|---|---|---|
| (° C.) | (g/h) | Catalyst | Activation | Composition | Time (sec) | Rate |
| 600 | 13.8 | 316 stainless | catalyst was | 37.33% 1132(E), | 1 | 37.33% |
| | | steel Propack | heated to | 62.23% 1132(Z), | | |
| | | | 350 C. under | 0.46% others | | |
| | | | 20 ml/min N2 | | | |
| | | | atmosphere | | | |
| 600 | 27.6 | 316 stainless | catalyst | 37.69% 1132(E), | 0.5 | 37.69% |
| | | steel Propack | reused from | 61.67% 1132(Z), | | |
| | | | above | 0.64% others | | |
| 550 | 29.3 | 316 stainless | catalyst | 29.98% 1132(E), | 0.5 | 29.98% |
| | | steel Propack | reused from | 69.69% 1132(Z), | | |
| | | | above | 0.33% others | | |
| 500 | 31.2 | 316 stainless | catalyst | 18.68% 1132(E), | 0.5 | 18.68% |
| | | steel Propack | reused from | 81.24% 1132(Z), | | |
| | | | above | 0.64% others | | |
| 500 | 5.2 | 316 stainless | catalyst | 32.99% 1132(E), | 3 | 32.99% |
| | | steel Propack | reused from | 66.54% 1132(Z), | | |
| | | | above | 0.47% others | | |
| 400 | 3.6 | 316 stainless | catalyst | 4.40% 1132(E), | 5 | 4.40% |
| | | steel Propack | reused from | 95.36% 1132(Z), | | |
| | | | above | 0.24% others | | |
| 350 | 3.9 | Fluorinated | catalyst | 0.04% 1,1- | 5 | 4.00% |
| | | aluminum | reused from | difluoroethylene, | | |
| | | oxide | above | 0.02% 1,1,1- | | |
| | | | | trifluoroethane, | | |
| | | | | 4.00% 1132(E), | | |
| | | | | 95.84% 1132(Z), | | |
| | | | | 0.10% 1,1,2- | | |
| | | | | trifluoroethane, | | |
| | | | | 0.01% others | | |
| 400 | 17.9 | Fluorinated | catalyst | 0.03% 1,1- | 1 | 4.22% |
| | | aluminum | reused from | difluoroethylene, | | |
| | | oxide | above | 4.22% 1132(E), | | |
| | | | | 95.62% 1132(Z), | | |
| | | | | 0.14% 1,1,2- | | |
| | | | | trifluoroethane. | | |
| 350 | 19.4 | fluorinated | catalyst was | 0.05% ethane, | 1 | 7.89% |
| | | chromium | slowly heated | 0.13% 1,1- | | |
| | | oxide | at 2 C./min to | difluoroethylene, | | |
| | | | 280 C. under | 0.11% 1,1,1- | | |
| | | | 100 ml/min air | tifluoroethane, | | |
| | | | atmosphere | 0.03% | | |
| | | | for 2 h, then | fluoroethylene, | | |
| | | | 350 C. for 4 h | 7.89% 1132(E), | | |
| | | | before use | 91.08% 1132(Z), | | |
| | | | | 0.68% 1,1,2- | | |
| | | | | tirfluoroethane, | | |
| | | | | 0.05% others | | |
| 350 | 18.5 | fluorinated | catalyst | 0.12% 1,1- | 3 | 13.49% |
| | | chromium | reused from | difluoroethylene, | | |
| | | oxide | above | 0.26% 1,1,1- | | |
| | | | | trifluoroethane, | | |
| | | | | 0.02% | | |
| | | | | fluoroethylene, | | |
| | | | | 13.49% 1132(E), | | |
| | | | | 85.39% 1132(Z), | | |
| | | | | 0.68% 1,1,2- | | |
| | | | | trifluoroethane, | | |
| | | | | 0.03% others | | |
| 380 | 5.2 | fluorinated | catalyst | 0.17% 1,1- | 1 | 7.42% |
| | | chromium | reused from | difluoroethylene, | | |
| | | oxide | above | 0.11% 1,1,1- | | |
| | | | | trifluoroethane, | | |
| | | | | 0.01% | | |
| | | | | fluoroethylene, | | |
| | | | | 7.42% 1132(E), | | |
| | | | | 91.95% | | |
| | | | | 1132(Z),0.30% | | |
| | | | | 1,1,2- | | |
| | | | | trifluoroethane, | | |
| | | | | 0.04% others | | |
| 380 | 36.9 | fluorinated | catalyst | 0.08% 1,1- | 0.5 | 3.30% |
| | | chromium | reused from | difluoroethylene, | | |
| | | oxide | above | 0.04% 1,1,1- | | |
| | | | | trifluoroethane, | | |
| | | | | 0.01% | | |
| | | | | fluoroethylene, | | |
| | | | | 3.30% 1132(E), | | |
| | | | | 96.28% 1132(Z), | | |
| | | | | 0.23% 1,1,2- | | |
| | | | | trifluoroethane, | | |
| | | | | 0.06% unidentified | | |
| | | | | minor product | | |
| 500 | 15.6 | Nickel Alloy | catalyst was | 34.33% 1132(E), | 1 | 34.33% |
| | | 200 Propack | heated to | 65.57% 1132(Z), | | |
| | | (99.6% Ni) | 350 C. under | 0.10% others | | |
| | | | 20 ml/min N2 | | | |
| | | | atmosphere | | | |
| | | | for 4 h before | | | |
| | | | use | | | |
| 500 | 31.2 | Nickel Alloy | catalyst | 26.76% 1132(E), | 0.5 | 26.76% |
| | | 200 Propack | reused from | 73.02% 1132(Z), | | |
| | | (99.6% Ni) | above | 0.22% others | | |
| 400 | 3.6 | Nickel Alloy | catalyst | 13.41% | 5 | 13.41% |
| | | 200 Propack | reused from | 1132(E),86.49% | | |
| | | (99.6% Ni) | above | | | |
| | | | | 1132(Z), 0.10% | | |
| | | | | others | | |
| 500 | 5.2 | Nichrome | catalyst was | 29.66% 1132(E), | 1 | 29.66% |
| | | Propack | heated to | 70.29% 1132(Z), | | |
| | | (Manganese | 350 C. under | 0.05% others | | |
| | | 1.5%, Nickel | 20 ml/min N2 | | | |
| | | 75% to 78%, | atmosphere | | | |
| | | Chromium | for 4 h before | | | |
| | | 20% to 23%, | use | | | |
| | | and a little | | | | |
| | | percentage of | | | | |
| | | Iron) | | | | |
| 650 | 15.5 | 5.14ml Nickel | catalyst | 23.65% 1132(E), | 1.01 | 23.65% |
| | | Alloy 200 | reused from | 72.64% 1132(Z), | | |
| | | Propack | above | 3.71% others | | |
| | | (99.6% Ni) | | | | |

### Example 2

### Isomerization of 1132(Z) to 1132(E) (Single Pass Through an Autoclave Reactor)

The process was carried out as described above and shown in FIG. 1 under conditions included in Table 22 below. The autoclave reactor had a volume of about 600 ml. The autoclave reactor was dried and pressure tested to 500 psig for integrity before any experiment was performed. A flow of N₂ in the autoclave reactor was vented in the hood, and any solid catalysts (e.g., iodine crystals) were preloaded before the pressure check. The autoclave reactor was vacuumed to 1 mbar and cooled down to −78° C. with a dry-ice bath, and the pre-weighed amount of reactant composition was then be condensed into the autoclave reactor and sealed.

The feed stream (reactant composition) included 99.8% cis-1,2-difluoroethene (1132(Z)). The contact time was 0.5 hour. The conversion rate of 1132(Z) to 1132(E) after a single pass through the reactor was from about 12.62% to about 40.15%, based on total amount of organic components of the product composition. The selectivity to desired product (1132(E)) was from about 98.88% to about 99.79%, based on total moles of organic components of the composition. The reaction may be repeated in an integrated process to form an end product including an amount of 1132(E) of greater than about 90% and less than or equal to 100%, or greater than about 95% and less than or equal to 100%, based on total moles of organic components of the composition.

TABLE 22
Process conditions and product compositions for a single pass through an
autoclave reactor in Example 2

| T | Pressure | | Catalyst | Product | Selectivity | Conversion |
|---|---|---|---|---|---|---|
| (° C.) | (psig) | Catalyst | Activation | Composition | to 1132(E) | Rate |
| 350 | 78 | iodine | none | 39.50% | 98.88% | 39.50% |
| | | crystals | | 1132(E), | | |
| | | | | 59.38% | | |
| | | | | 1132(Z), | | |
| | | | | 1.12% others | | |
| 300 | 75 | iodine | none | 40.15% | 99.13% | 40.15% |
| | | crystals | | 1132(E), | | |
| | | | | 58.98% | | |
| | | | | 1132(Z), | | |
| | | | | 0.87% others | | |
| 350 | 100 | none | none | 12.62% | 99.79% | 12.62% |
| | | | | 1132(E), | | |
| | | | | 87.17% | | |
| | | | | 1132(Z), | | |
| | | | | 0.87% others | | |
| 300 | 68 | iodine | none | 36.64% | 99.65% | 36.64% |
| | | crystals | | 1132(E), | | |
| | | | | 63.01% | | |
| | | | | 1132(Z), | | |
| | | | | 0.35% others | | |

### Example 3

### Isomerization of 1132(E) to 1132Z (Single Pass Through a Tubular Reactor)

The process was carried out as described above and shown in FIG. 1 under conditions included in Table 23 below. The feed stream (reactant composition) included 98.07% trans-1,2-difluoroethene (1132(E)) and 0.93% cis-1,2-difluoroethene (1132(Z)). The conversion rate of 1132(E) to 1132(Z) after a single pass through the reactor was from about 0.6% to about 50.41%, based on total moles of organic components of the composition. The selectivity to desired product (1132(Z)) was from about 91.19% to about 99.26%, based on total moles of organic components of the composition. The reaction may be repeated in an integrated process to form an end product including an amount of 1132(Z) of greater than about 90% and less than or equal to 100%, or greater than about 95% and less than or equal to 100%, based on total moles of organic components of the composition.

TABLE 23
Process conditions and product compositions for a single pass through a
tubular reactor in Example 3

| Flow | | | Reactor | Contact | | | | |
|---|---|---|---|---|---|---|---|---|
| Rate | | P | Volume | Time | | Catalyst | Product | Conversion |
| (g/h) | T (° C.) | (psig) | (ml) | (sec) | Catalyst | Activation | Composition | rate |
| 11.0 | 600 | 15 | 17.14 | 5 | 316 | catalyst was | 68.19% | 27.43% |
| | | | | | stainless | heated to | 1132(E), | |
| | | | | | steel | 350 C. under | 27.43% | |
| | | | | | Propack | 20 ml/min N2 | 1132(Z), | |
| | | | | | | atmosphere | 4.38% other | |
| 24.0 | 650 | 15 | 8.57 | 1.1 | 316 | catalyst | 74.32% | 24.05% |
| | | | | | stainless | reused from | 1132(E),24.05 | |
| | | | | | steel | above | % 1132(Z), | |
| | | | | | Propack | | 1.63% other | |
| 24.0 | 700 | 15 | 8.57 | 1 | 316 | catalyst | 40.78% | 50.41% |
| | | | | | stainless | reused from | 1132(E), | |
| | | | | | steel | above | 50.41% | |
| | | | | | Propack | | 1132(Z), | |
| | | | | | | | 8.81% other | |
| 7.7 | 350 | 15 | 17.14 | 10 | fluorinated | catalyst was | 84.72% | 13.98% |
| | | | | | aluminum | heated to | 1132(E), | |
| | | | | | oxide | 350 C. under | 13.98% | |
| | | | | | | 20 ml/min N2 | 1132(Z), | |
| | | | | | | atmosphere | 1.30% other | |
| | | | | | | for 4 h | | |
| | | | | | | before use | | |
| 7.1 | 400 | 15 | 17.14 | 10 | fluorinated | catalyst | 56.19% | 42.14% |
| | | | | | aluminum | reused from | 1132(E), | |
| | | | | | oxide | above | 42.14% | |
| | | | | | | | 1132(Z), | |
| | | | | | | | 1.67% other | |
| 14.3 | 400 | 15 | 17.14 | 5 | fluorinated | catalyst | 83.72% | 15.27% |
| | | | | | aluminum | reused from | 1132(E), | |
| | | | | | oxide | above | 15.27% | |
| | | | | | | | 1132(Z), | |
| | | | | | | | 1.01% other | |
| 24.0 | 350 | 45 | 17.14 | 3.1 | fluorinated | catalyst | 90.72% | 8.12% |
| | | | | | aluminum | reused from | 1132(E), | |
| | | | | | oxide | above | 8.12% | |
| | | | | | | | 1132(Z), | |
| | | | | | | | 1.16% other | |
| 12.4 | 500 | 15 | 17.14 | 5 | Nickel alloy | catalyst was | 95.94% | 1.85% |
| | | | | | 200 (99.6% | heated to | 1132(E), | |
| | | | | | Ni) | 350 C. under | 1.85% | |
| | | | | | | 20 ml/min N2 | 1132(Z), | |
| | | | | | | atmosphere | 2.21% other | |
| | | | | | | for 4 h | | |
| | | | | | | before use | | |
| 24.0 | 400 | 45 | 17.14 | 3.1 | Nickel alloy | catalyst | 94.31% | 4.20% |
| | | | | | 200 (99.6% | reused from | 1132(E), | |
| | | | | | Ni) | above | 4.20% | |
| | | | | | | | 1132(Z), | |
| | | | | | | | 1.49% other | |
| 12.7 | 350 | 15 | 8.57 | 3 | fluorinated | catalyst was | 72.91% | 23.83% |
| | | | | | chromium | heated to | 1132(E), | |
| | | | | | oxide | 280 C. under | 23.83% | |
| | | | | | | 100 ml/min | 1132(Z), | |
| | | | | | | air | 3.26% other | |
| | | | | | | atmosphere | | |
| | | | | | | for 2 h then | | |
| | | | | | | 350 C. for 4 h | | |
| | | | | | | before use | | |
| 24.0 | 400 | 15 | 8.57 | 3 | fluorinated | catalyst | 0.79% 1,1,1- | 27.56% |
| | | | | | chromium | reused from | trifluoroethan, | |
| | | | | | oxide | above | 70.42% | |
| | | | | | | | 1132(E), | |
| | | | | | | | 27.56% | |
| | | | | | | | 1132(Z), | |
| | | | | | | | 1.23% other | |

### Example 4

### Isomerization of 1132(E) to 1132(Z) (Single Pass Through an Autoclave Reactor)

The process was carried out as described above and shown in FIG. 1 under conditions included in Table 24 below. The feed stream (reactant composition) included 98.07% trans-1,2-difluoroethene (1132(E)) and 0.93% cis-1,2-difluoroethene (1132(Z)). The reactor used is a stainless steel autoclave reactor had a volume of 600 ml. The conversion rate of 1132(E) to 1132(Z) after a single pass through the reactor was from about 0.14% to about 69.91%, based on total moles of organic components of the composition. The selectivity to desired product (1132(Z)) was from about 98.94% to about 99.90%, based on total moles of organic components of the composition. The reaction may be repeated in an integrated process to form an end product including an amount of 1132(Z) of greater than about 90% and less than or equal to 100%, or greater than about 95% and less than or equal to 100%, based on total moles of organic components of the composition.

TABLE 24
Process conditions and product compositions for a single pass through an
autoclave reactor in Example 4

| | | Contact | | | Selectivity | |
|---|---|---|---|---|---|---|
| Temp (° C.) | P (psig) | Time (h) | Catalyst | Product Composition | to 1132(Z) | Conversion Rate |
| 100 | 49 | 2 | 0.5 g | 98.10% 1132(E), | 99.26% | 1.16% |
| | | | iodine | 1.16% 1132(Z), 0.74% | | |
| | | | crystals | other | | |
| 250 | 79 | 2.5 | 0.5 g | 38.53% 1132(E), | 99.15% | 60.62% |
| | | | iodine | 60.62% 1132(Z), | | |
| | | | crystals | 0.85% other | | |
| 250 | 79 | 1 | 0.5 g | 42.03% 1132(E), | 99.27% | 57.24% |
| | | | iodine | 57.24% 1132(Z), | | |
| | | | crystals | 0.73% other | | |
| 300 | 67 | 0.5 | 0.5 g | 31.65% 1132(E), | 99.39% | 67.74% |
| | | | iodine | 67.74% 1132(Z), | | |
| | | | crystals | 0.74% other | | |
| 350 | 50 | 0.5 | 0.5 g | 33.69% 1132(E), | 99.31% | 65.62% |
| | | | iodine | 65.62% 1132(Z), | | |
| | | | crystals | 0.69% other | | |
| 250 | 340 | 1 | none | 99.76% 1132(E), | 99.90% | 0.14% |
| | | | | 0.14% 1132(Z), 0.10% | | |
| | | | | other | | |
| 300 | 310 | 0.5 | none | 99.52% 1132(E), | 99.82% | 0.30% |
| | | | | 0.30% 1132(Z), 0.18% | | |
| | | | | other | | |
| 350 | 317 | 0.5 | none | 97.67% 1132(E), | 99.09% | 1.42% |
| | | | | 1.42% 1132(Z), 0.81% | | |
| | | | | other | | |
| 300 | 332 | 0.5 | 0.11 g | 93.05% 1132(E), | 99.84% | 6.79% |
| | | | iodine | 6.79% 1132(Z), 0.16% | | |
| | | | crystal | other | | |
| 350 | 382 | 0.5 | 0.12 g | 40.53% 1132(E), | 98.94% | 58.41% |
| | | | iodine | 58.41% 1132(Z), | | |
| | | | crystal | 1.06% other | | |
| 350 | 382 | 0.5 | 0.5 g | 36.96% 1132(E), | 98.99% | 62.03% |
| | | | iodine | 62.03% 1132(Z), | | |
| | | | crystals | 1.01% other | | |
| 350 | 150 | 0.5 | 0.11 g | 29.08% 1132(E), | 98.99% | 69.91% |
| | | | iodine | 69.91% 1132(Z), | | |
| | | | crystal | 1.01% other | | |

### Example 5

### Isomerization of 1132(Z) to 1132(E) (Single Pass Through a Tubular Reactor Using a Mixture of 1132(Z) and 1132(E) as Feed Stream)

The process is carried out as described above and shown in FIG. 1 under similar conditions as described in Example 1. The feed stream (reactant composition) includes from about 91.0% to about 93.3% 1132(Z) and from about 6.7% to about 9.0% 1132(E). The selectivity to desired product (1132(Z)) is from about 98.94% to about 99.90%, based on total moles of organic components of the composition. The reaction may be repeated in an integrated process to form an end product including an amount of 1132(Z) of greater than about 90% and less than or equal to 100%, or greater than about 95% and less than or equal to 100%, based on total moles of organic components of the composition.

### Example 6

### Isomerization of 1132(Z) to 1132(E) (Single Pass Through an Autoclave Reactor Using a Mixture of 1132(Z) and 1132(E) as Feed Stream)

The process is carried out as described above and shown in FIG. 1 under similar conditions as described in Example 2. The feed stream (reactant composition) includes from about 91.0% to about 93.3% 1132(Z) and from about 6.7% to about 9.0% 1132(E). The selectivity to desired product (1132(Z)) is from about 98.94% to about 99.90%, based on total moles of organic components of the composition. The reaction may be repeated in an integrated process to form an end product including an amount of 1132(Z) of greater than about 90% and less than or equal to 100%, or greater than about 95% and less than or equal to 100%, based on total moles of organic components of the composition.

### Example 7

### Isomerization of 1132(E) to 1132(Z) (Single Pass Through a Tubular Reactor Using a Mixture of 1132(Z) and 1132(E) as Feed Stream)

The process is carried out as described above and shown in FIG. 1 under similar conditions as described in Example 3. The feed stream (reactant composition) includes from about 91.0% to about 93.3% 1132(Z) and from about 6.7% to about 9.0% 1132(E). The selectivity to desired product (1132(Z)) is from about 98.94% to about 99.90%, based on total moles of organic components of the composition. The reaction may be repeated in an integrated process to form an end product including an amount of 1132(Z) of greater than about 90% and less than or equal to 100%, or greater than about 95% and less than or equal to 100%, based on total moles of organic components of the composition.

### Example 8

### Isomerization of 1132(Z) to 1132(E) (Single Pass Through a Tubular Reactor Using a Mixture of 1132(Z) and 1132(E) as Feed Stream)

The process is carried out as described above and shown in FIG. 1 under similar conditions as described in Example 4. The feed stream (reactant composition) includes from about 91.0% to about 93.3% 1132(Z) and from about 6.7% to about 9.0% 1132(E). The selectivity to desired product (1132(Z)) is from about 98.94% to about 99.90%, based on total moles of organic components of the composition. The reaction may be repeated in an integrated process to form an end product including an amount of 1132(Z) of greater than about 90% and less than or equal to 100%, or greater than about 95% and less than or equal to 100%, based on total moles of organic components of the composition.

### ASPECTS

Aspect 1 is a method for producing trans-1,2-difluoroethylene (HFO-1132(E)), comprising: providing a reactant composition comprising cis-1,2-difluoroethylene (HFO-1132(Z)); and isomerizing the cis-1,2-difluoroethylene (HFO-1132(Z)) in a reactor at a temperature of from about 300° C. to about 700° C. in the presence of a catalyst to produce a product composition comprising HFO-1132(E).

Aspect 2 is the method of Aspect 1, wherein the isomerizing step is conducted as a continuous process through a tubular reactor.

Aspect 3 the method of Aspect 1 or Aspect 2, wherein the catalyst is selected from the group consisting of stainless steel, fluorinated aluminum oxide, fluorinated chromium oxide, a metal alloy comprising nickel, chromium, manganese, iron, and a combination thereof.

Aspect 4 is the method of any one of Aspects 1-3, wherein the isomerizing step is conducted at a temperature of from about 400° C. to about 650° C.

Aspect 5 is the method of any one of Aspects 1-4, wherein the isomerizing step is conducted at a pressure of from about 10 psig to about 30 psig.

Aspect 6 is the method of any one of Aspects 1-5, wherein the isomerizing step is conducted with a contact time of the reactant composition with the catalyst of from about 0.1 seconds to about 10 seconds.

Aspect 7 is the method of any one of Aspects 1-6, further comprising, prior to the isomerizing step: activating the catalyst by heating to a temperature of from about 280° C. to about 350° C.

Aspect 8 is the method of any one of Aspects 1-7, wherein the isomerizing step further comprises a contact time of the reactant composition with the catalyst of from about 0.1 seconds to about 5 seconds.

Aspect 9 is the method of any one of Aspects 1-8, wherein: the catalyst comprises stainless steel; the isomerizing step is conducted at a temperature of from about 500° C. to about 600° C., at atmospheric pressure, and a contact time of from about 0.1 second to about 5 seconds; and the product composition comprises from about 20 wt. % to about 50 wt. % of HFO-1132(E) and from about 50 wt. % to about 80 wt. % of HFO-1132(Z) after a single pass through the tubular reactor, based on the total amount of organic components of the product composition.

Aspect 10 is the method of any one of Aspects 1-8, wherein: the catalyst comprises a nickel alloy; the isomerizing step is conducted at a temperature of from about 450° C. to about 550° C., at atmospheric pressure, and a contact time of from about 0.1 second to about 5 seconds; and the product composition comprises from about 10 wt. % to about 50 wt. % of HFO-1132(E) and from about 50 wt. % to about 90 wt. % of HFO-1132(Z) after a single pass through the tubular reactor, based on the total amount of organic components of the product composition.

Aspect 11 is the method of any one of Aspects 1-8, wherein: the catalyst comprises nickel; the isomerizing step is conducted at a temperature of from about 450° C. to about 550° C. at atmospheric pressure; and the product composition comprises from about 10 wt. % to about 50 wt. % of HFO-1132(E) and from about 50 wt. % to about 90 wt. % of HFO-1132(Z) after a single pass through the tubular reactor, based on the total amount of organic components of the product composition.

Aspect 12 is the method of Aspect 1, wherein the isomerizing step is conducted in a semi-batch process within an autoclave reactor.

Aspect 13 is the method of Aspect 12, wherein the isomerizing step is conducted with a contact time of the reactant composition with the autoclave reactor of from about 0.5 hour to about 1 hour.

Aspect 14 is the method of Aspects 12 or 13, wherein the catalyst comprises iodine crystals

Aspect 15 is the method of any one of Aspects 12-14, wherein: the isomerizing step is conducted at a temperature of from about 300° C. to about 380° C. at a pressure of from about 68 to about 76 psig; and the product composition comprises from about 10% to about 50 wt. % of HFO-1132(E) and from about 50 wt. % to about 90 wt. % of HFO-1132(Z) after a single pass through the autoclave reactor, based on the total amount of organic components of the product composition.

Aspect 16 is a method for producing cis-1,2-difluoroethylene (HFO-1132(Z)), comprising: providing a reactant composition comprising trans-1,2-difluoroethylene (HFO-1132(E)); and isomerizing the trans-1,2-difluoroethylene (HFO-1132(E)) in a reactor at a temperature of from about 250° C. to about 700° C. in the presence of a catalyst to produce a product composition comprising HFO-1132(Z).

Aspect 17 is the method of Aspect 16, wherein the isomerizing step is conducted as a continuous process through a tubular reactor.

Aspect 18 is the method of Aspect 16 or Aspect 17, wherein the catalyst is selected from the group consisting of stainless steel, fluorinated aluminum oxide, fluorinated chromium oxide, and a metal alloy comprising nickel.

Aspect 19 is the method of any one of Aspects 16-18, wherein the isomerizing step is conducted at a temperature of from about 400° C. to about 700° C.

Aspect 20 is the method of any one of Aspects 16-19, wherein the isomerizing step is conducted at a pressure of from about 15 psig to about 45 psig.

Aspect 21 is the method of any one of Aspects 16-20, wherein the isomerizing step is conducted with a contact time of the reactant composition with the catalyst of from about 0.1 seconds to about 15 seconds.

Aspect 22 is the method of any one of Aspects 16-21, wherein the isomerizing step is conducted with a contact time of the reactant composition with the catalyst of from about 1 seconds to about 10 seconds.

Aspect 23 is the method of Aspect 16, wherein the isomerizing step is conducted in a semi-batch process within an autoclave reactor; wherein the catalyst is iodine crystals.

Aspect 24 is the method of Aspect 23, wherein the isomerizing step is conducted at a temperature of from about 250° C. to about 350° C.

Aspect 25 is the method of any one of Aspects 1-8, wherein the catalyst comprises stainless steel, wherein the isomerizing step is conducted at a temperature of from about 300° C. to about 800° C., a pressure of from about 1 psig to about 50 psig, with a contact time of the reactant composition with the catalyst of from about 0.1 seconds to about 10 seconds.

Aspect 26 is the method of any one of Aspects 1-8, wherein the catalyst comprises stainless steel, wherein the isomerizing step is conducted at a temperature of from about 500° C. to about 600° C., a pressure of from about 10 psig to about 20 psig, with a contact time of the reactant composition with the catalyst of from about 0.1 seconds to about 5 seconds.

Aspect 27 is the method of any one of Aspects 1-8, wherein the catalyst comprises fluorinated aluminum oxide, wherein the isomerizing step is conducted at a temperature of from about 300° C. to about 800° C., a pressure of from about 1 psig to about 50 psig, with a contact time of the reactant composition with the catalyst of from about 0.1 seconds to about 10 seconds.

Aspect 28 is the method of any one of Aspects 1-8, wherein the catalyst comprises fluorinated aluminum oxide, wherein the isomerizing step is conducted at a temperature of from about 350° C. to about 650° C., a pressure of from about 10 psig to about 20 psig, with a contact time of the reactant composition with the catalyst of from about 0.1 seconds to about 5 seconds.

Aspect 29 is the method of any one of Aspects 1-8, wherein the catalyst comprises 99% nickel, wherein the isomerizing step is conducted at a temperature of from about 300° C. to about 800° C., a pressure of from about 1 psig to about 50 psig, with a contact time of the reactant composition with the catalyst of from about 0.1 seconds to about 10 seconds.

Aspect 30 is the method of any one of Aspects 1-8, wherein the catalyst comprises 99% nickel, wherein the isomerizing step is conducted at a temperature of from about 450° C. to about 550° C., a pressure of from about 10 psig to about 20 psig, with a contact time of the reactant composition with the catalyst of from about 0.5 seconds to about 5 seconds.

Aspect 31 is the method of any one of Aspects 1-8, wherein the catalyst comprises a nickel alloy, wherein the isomerizing step is conducted at a temperature of from about 300° C. to about 800° C., a pressure of from about 1 psig to about 50 psig, with a contact time of the reactant composition with the catalyst of from about 0.1 seconds to about 10 seconds.

Aspect 32 is the method of any one of Aspects 1-8, wherein the catalyst comprises a nickel alloy, wherein the isomerizing step is conducted at a temperature of from about 450° C. to about 550° C., a pressure of from about 10 psig to about 20 psig, with a contact time of the reactant composition with the catalyst of from about 0.1 seconds to about 5 seconds.

Aspect 33 is the method of any one of Aspects 12-13, wherein the catalyst comprises a iodine crystals, wherein the isomerizing step is conducted at a temperature of from about 50° C. to about 450° C., a pressure of from about 60 psig to about 120 psig, with a contact time of the reactant composition with the catalyst of from about 0.1 hour to about 1 hour.

Aspect 34 is the method of any one of Aspects 12-13, wherein the catalyst comprises a iodine crystals, wherein the isomerizing step is conducted at a temperature of from about 100° C. to about 380° C., a pressure of from about 65 psig to about 100 psig, with a contact time of the reactant composition with the catalyst of from about 0.4 hour to about 0.6 hour.

Aspect 35 is the method of any one of Aspects 12-13, wherein the isomerizing step is conducted without use of a catalyst or irradiation, at a temperature of from about 50° C. to about 450° C., a pressure of from about 60 psig to about 120 psig, with a contact time of the reactant composition with the autoclave reactor of from about 0.1 hour to about 1 hour.

Aspect 36 is the method of any one of Aspects 12-13, wherein the isomerizing step is conducted without use of a catalyst or irradiation, at a temperature of from about 100° C. to about 380° C., a pressure of from about 65 psig to about 100 psig, with a contact time of the reactant composition with the autoclave reactor of from about 0.4 hour to about 0.6 hour.

Aspect 37 is the method of any one of Aspects 17-18, wherein the product composition comprises from about 25 wt. % to about 80 wt. % of HFO-1132(Z) and from about 20 wt. % to about 75 wt. % of HFO-1132(E) after a single pass through the tubular reactor, based on the total amount of organic components of the product composition.

Aspect 38 is the method of any one of Aspects 23-24, wherein the product composition comprises from about 50 wt. % to about 80 wt. % of HFO-1132(Z) and from about 20 wt. % to about 50 wt. % of HFO-1132(E) after a single pass through the autoclave reactor, based on the total amount of organic components of the product composition.

It should be understood that the foregoing description is only illustrative of the present disclosure. Various alternatives and modifications can be devised by those skilled in the art without departing from the disclosure. Accordingly, the present disclosure is intended to embrace all such alternatives, modifications and variances that fall within the scope of the appended claims.

1. A method for producing trans-1,2-difluoroethylene (HFO-1132(E)), comprising:
providing a reactant composition comprising cis-1,2-difluoroethylene (HFO-1132(Z)); and
isomerizing the cis-1,2-difluoroethylene (HFO-1132(Z)) in a reactor at a temperature of from about 300° C. to about 700° C. in the presence of a catalyst to produce a product composition comprising HFO-1132(E).

2. The method of claim 1, wherein the isomerizing step is conducted as a continuous process through a tubular reactor.

3. The method of claim 1, wherein the catalyst is selected from the group consisting of stainless steel, fluorinated aluminum oxide, fluorinated chromium oxide, a metal alloy comprising nickel, chromium, manganese, iron, and a combination thereof.

4. The method of claim 1, wherein the isomerizing step is conducted at a temperature of from about 400° C. to about 650° C.

5. The method of claim 1, wherein the isomerizing step is conducted at a pressure of from about 10 psig to about 30 psig.

6. The method of claim 1, wherein the isomerizing step is conducted with a contact time of the reactant composition with the catalyst of from about 0.1 seconds to about 10 seconds.

7. The method of claim 1, further comprising, prior to the isomerizing step:
activating the catalyst by heating to a temperature of from about 280° C. to about 350° C.

8. The method of claim 1, wherein the isomerizing step further comprises a contact time of the reactant composition with the catalyst of from about 0.1 seconds to about 5 seconds.

9. The method of claim 1, wherein:
the catalyst comprises stainless steel;
the isomerizing step is conducted at a temperature of from about 500° C. to about 600° C., at atmospheric pressure, and a contact time of from about 0.1 second to about 5 seconds; and
the product composition comprises from about 20 wt. % to about 50 wt. % of HFO-1132(E) and from about 50 wt. % to about 80 wt. % of HFO-1132(Z) after a single pass through the tubular reactor, based on total amount of organic components of the product composition.

10. The method of claim 1, wherein:
the catalyst comprises a nickel alloy;
the isomerizing step is conducted at a temperature of from about 450° C. to about 550° C., at atmospheric pressure, and a contact time of from about 0.1 second to about 5 seconds; and
the product composition comprises from about 10 wt. % to about 50 wt. % of HFO-1132(E) and from about 50 wt. % to about 90 wt. % of HFO-1132(Z) after a single pass through the tubular reactor, based on total amount of organic components of the product composition.

11. The method of claim 1, wherein:
the catalyst comprises nickel;
the isomerizing step is conducted at a temperature of from about 450° C. to about 550° C. at atmospheric pressure; and
the product composition comprises from about 10 wt. % to about 50 wt. % of HFO-1132(E) and from about 50 wt. % to about 90 wt. % of HFO-1132(Z) after a single pass through the tubular reactor, based on total amount of organic components of the product composition.

12. The method of claim 1, wherein the isomerizing step is conducted in a semi-batch process within an autoclave reactor.

13. The method of claim 1, wherein the isomerizing step is conducted with a contact time of the reactant composition with the autoclave reactor of from about 0.5 hour to about 1 hour.

14. The method of claim 1, wherein the catalyst comprises iodine crystals.

15. The method of claim 1, wherein:
the isomerizing step is conducted at a temperature of from about 300° C. to about 380° C. at a pressure of from about 68 to about 76 psig; and
the product composition comprises from about 10% to about 50 wt. % of HFO-1132(E) and from about 50 wt. % to about 90 wt. % of HFO-1132(Z) after a single pass through the autoclave reactor, based on total amount of organic components of the product composition.

16. A method for producing cis-1,2-difluoroethylene (HFO-1132(Z)), comprising:
providing a reactant composition comprising trans-1,2-difluoroethylene (HFO-1132(E)); and
isomerizing the trans-1,2-difluoroethylene (HFO-1132(E)) in a reactor at a temperature of from about 250° C. to about 700° C. in the presence of a catalyst to produce a product composition comprising HFO-1132(Z).

17. The method of claim 1, wherein the isomerizing step is conducted as a continuous process through a tubular reactor.

18. The method of claim 1, wherein the catalyst is selected from the group consisting of stainless steel, fluorinated aluminum oxide, fluorinated chromium oxide, and a metal alloy comprising nickel.

19. The method of claim 1, wherein the isomerizing step is conducted at a temperature of from about 400° C. to about 700° C.

20. The method of claim 1, wherein the isomerizing step is conducted at a pressure of from about 15 psig to about 45 psig.
